(19) United States
(12) Patent Application Publication
(10) Pub. No.: US 2025/0354192 A1
(43) Pub. Date: Nov. 20, 2025

(54) METHODS FOR QUANTIFYING LIVE BACTERIA

(71) Applicant: ANCERA INC., Branford, CT (US)

(72) Inventors: Anastasia K. SEVOSTIYANOVA, North Haven, CT (US); Sylvia L. RIVERA, Middletown, CT (US); Margaret M. KISS, Westbrook, CT (US)

(21) Appl. No.: 18/874,133

(22) PCT Filed: Jun. 16, 2023
(86) PCT No.: PCT/US2023/068592

§ 371 Date: Dec. 12, 2024

Related U.S. Application Data
(60) Provisional application No. 63/353,447, filed on Jun. 17, 2022.

(51) Int. Cl. C12Q 1/06

(52) U.S. Cl. CPC C12Q 1/06

(57) ABSTRACT

The present disclosure provides methods, systems, and devices for the in situ labeling of complex environmental samples, specifically mixed populations of total live bacteria, using D-amino acid analogs, such as fluorescent D-amino acids or D-amino acids with a biorthogonal tag to enable chemical conjugation of a fluorophore (e.g. by click chemistry). The disclosure further provides methods for quantifying the TVB by cell manipulation using ferrofluid within a microfluidic device.

### CROSS REFERENCE TO RELATED APPLICATIONS

This application claims priority to and the benefit of U.S. Provisional Application No. 63/353,447, filed on Jun. 17, 2022, which is incorporated by reference herein in its entirety for all purposes.

### RELATED APPLICATIONS

The current disclosure is related to U.S. Pat. Nos. 8,961,878, and 9,999,855, and PCT publication nos. WO2014144340, WO2014144782, WO2014144810, WO2014145765, WO2014165317, WO2016210348, WO2017004595, WO2018026605, WO2019117877, and WO2022/015845. Each of the foregoing disclosures is herein incorporated by reference in its entirety.

### BACKGROUND

The standard method for quantification of total live bacteria is based on serial dilution followed by plating, and incubation of the plates (culturing) until countable macroscopic colonies are formed, which can take up to 48 hours. Substituting dehydrated medium contained in sheets or films for traditional agar plates can reduce sample preparation time and increase space efficiency, but these methods still rely on the formation of visible colonies, so the overall time savings is limited.

Alternative methods to traditional plate counts have been developed for early detection of bacterial growth either directly or indirectly (e.g., Soleris™ system supplied by the Neogen® Corporation which measures changes in pH and other biochemical indicators as bacteria grow), but since the results still rely on creating serial dilutions and culturing microorganisms present in the sample, the time to result remains at least 18-24 hours.

New methods for the analysis and quantification of total live bacteria remains a currently unmet need which is addressed by embodiments of the present disclosure.

### SUMMARY OF SOME OF THE EMBODIMENTS

The present disclosure is directed to methods, systems, and devices providing a single-cell alternative to CFU (colony forming unit) counts that (at least one of and preferably all of):
eliminates the waiting period required for development of macroscopic colonies;
offers a wide dynamic range of quantitation, therefore reducing or eliminating the need for serial dilutions; and
is user friendly due to most steps in the cell manipulation and counting being fully automated. With this method, results can be obtained in less than 3 hours.

Accordingly, in some embodiments, a method for quantifying total live bacteria in a sample is provided and includes obtaining a sample comprising a mixture of two or more types of live bacteria, and contacting the live bacteria with at least one D-amino acid probe under conditions sufficient for bacterial cell wall synthesis. The bacteria covalently incorporates the at least one D-amino acid probe, and the amino acid probe includes a covalently attached fluorophore. The method further includes adding the bacteria including the covalently incorporated D-amino acid probe to an assay processing device for at least counting cells, and detecting the labeled bacteria.

In some embodiments, such as those set out above (as well as other disclosed herein), may also include one and/or another of (and in some embodiments, a plurality of, in some embodiments, a majority of, in some embodiments, substantially all of, and in some embodiments, if not mutually exclusive, all of) the following features, structures, functionalities, steps, and clarifications, leading to yet further embodiments:
the D-amino acid probe is a single amino acid or a dipeptide;
removing unincorporated D-amino acid probes from the sample;
the labeled bacteria are detected via a fluorescence detector;
flowing the bacteria labeled with the D-amino acid probe toward an imaging region of the assay processing device, the imaging region including the fluorescence detector;
quantifying a number of total live bacteria detected in the sample; and
the D-amino acid probe is selected from the group consisting of: HADA, BADA, NADA, FDL, TDL, HDL, NDL, FADA, TADA, HADG, NADG, FADG, and TADG.

In some embodiments, a method for quantifying total live bacteria in a sample is provided and includes obtaining a sample comprising live bacteria, and contacting the live bacteria with at least one D-amino acid probe under conditions sufficient for bacterial cell wall synthesis. The bacteria covalently incorporate the at least one D-amino acid probe, and the D-amino acid probe comprises a clickable bioorthogonal handle. The method further includes contacting the live bacteria with a fluorescent label comprising a bioorthogonal reactive group, wherein the clickable bioorthogonal reactive group forms a covalent bond with the clickable bioorthogonal handle, and detecting the live bacteria.

In some embodiments, such as those set out above (as well as other disclosed herein), may also include one and/or another of (and in some embodiments, a plurality of, in some embodiments, a majority of, in some embodiments, substantially all of, and in some embodiments, if not mutually exclusive, all of) the following features, structures, functionalities, steps, and clarifications, leading to yet further embodiments:
the D-amino acid probe contains an azide, alkyne, or cycloalkyne group;
removing the unincorporated D-amino acid probe from the sample;
the labeled bacteria are detected via a fluorescence detector;
flowing the labeled bacteria including the at least one D-amino acid probe toward an imaging region of an assay device, the imaging region including the fluorescence detector;
quantifying the number of total live bacteria detected in the sample;
the D-amino acid probe includes an alkyne group;
the D-amino acid probe is D-propargylglycine (EDA);
the fluorescent label includes an azide, alkyne, or cycloalkyne group;
the fluorescent label includes an azide group;
the fluorescent label comprises CF488 picolyl azide or AZDye633 picolyl azide;
the clickable bioorthogonal handle of the D-amino acid probe reacts with the clickable bioorthogonal reactive group of the fluorescent label to form a 1,2,3-triazole;
the reaction between the D-amino acid and the fluorescent label is a copper catalyzed click chemistry reaction;
the reaction between the D-amino acid and the fluorescent label is a copper-free strain promoted click chemistry reaction;
the contacting the live bacteria with at least one D-amino acid probe comprises incubating the live bacteria with the at least one D-amino acid probe;
the contacting the live bacteria with at least one D-amino acid probe occurs for between about 10 and about 120 minutes;
the contacting the live bacteria with at least one D-amino acid probe occurs for at least about 10 minutes, at least about 20 minutes, at least about 30 minutes, at least about 40 minutes, at least about 50 minutes, at least about 60 minutes, at least about 70 minutes, at least about 80 minutes, at least about 90 minutes, at least about 100 minutes, at least about 110 minutes, or at least about 120 minutes;
the contacting the live bacteria with at least one D-amino acid probe occurs for between about 20 and about 90 minutes;
the contacting the live bacteria with at least one D-amino acid probe comprises incubating the live bacteria with the at least one D-amino acid probe for between about 20 and about 90 minutes;
the sample is supplemented with a cell metabolism booster, which can be at least one of glucose and sodium pyruvate;
the sample is supplemented with a small molecule that controls cell doubling, where the small molecule can be DL-serine hydroxamate or chloramphenicol;
the sample is supplemented with at least one of glucose and sodium pyruvate;
quantifying the total live bacteria in the sample is performed using a microfluidic device;
quantifying the total live bacteria in the sample is performed using flow cytometry;
quantifying the total live bacteria in the sample is performed using a ferrofluid-based microfluidic device;
the sample is obtained from a food processing plant;
the sample is obtained from a sample from a beef or poultry processing plant;
the sample is obtained from a poultry processing plant;
culturing the bacteria before analysis;
not including culturing the bacteria before analysis;
the quantifying step comprises counting individually labeled bacteria cells;
the live bacteria sample comprises gram negative bacteria;
the live bacteria is selected from the group consisting of: Aeromonas hydrophila, Bukholderia cenocepacia, Campylobacter jejuni, Citrobacter freundii, Enterobacter sakasakii (Cronobacter), Escherichia coli, Flavobacterium Sp., Hafnia alvei, Klebsiella pneumoniae, Kluyvera Sp., Moraxella catarrhalis, Proteus mirabilis, Pseudomonas aeruginosa, Salmonella Typhimurium, Serratia liquefaciens, Shewanella putrefaciens, Shigella flexneri, Vibrio parahaemolyticus, and Yersinia enterocolitica,
the live bacteria sample comprises gram positive bacteria;
the bacteria is selected from the group consisting of: Bacillus cereus, Bacteroides fragilis, Brochothrix thermosphacta, Clostridium perfringens, Corynebacterium sp, Enterococcus faecalis, Lactobacillus brevis, Lactococcus lactis, Leuconostoc lactis,
Listeria monocytogenes, Staphylococcus aureus, Streptococcus pyogenes, and Weissella viridescens; and
the sample comprises both gram negative and gram positive bacteria;

In some embodiments, a quantification system configured to quantify fluorescently labeled live bacteria from a sample according to any of the method embodiments set out above (or otherwise disclosed herein), the system including a ferrofluidic assay device configured to receive a microfluidic cartridge containing a sample, the microfluidic cartridge includes a plurality of microfluidic channels, each microfluidic channel contains an imaging window, an imager configured to image each window of the cartridge either separately or together, a controller configured to control at least one of the ferrofluidic assay device, the microfluidic cartridge containing sample mixed with ferrofluid, and the imager, and assay processing components comprising at least one of reagents, and controls. The system is configured to at least one of moving or otherwise locating the labeled bacteria to one or more of the windows where they can be any and all of imaged and quantified.

In some embodiments, the systems and methods described herein may be used to determine a number of live bacteria in a sample. The methods may be used, for example, to diagnose animals suspected to be infected with a bacteria. Specifically, the methods may be used to identify any livestock (e.g. flocks of poultry, etc.) at risk of decreased performance levels due to bacterial infection, and help in the development of treatment strategies.

The embodiments disclosed herein may also be used to assess the quality of various environmental conditions by rapidly determining total live bacteria.

The embodiments and corresponding inventions disclosed herein will become even more clear with reference to the drawings (a brief description of which is provided below) and detailed description and examples which follows.

### BRIEF DESCRIPTION OF THE DRAWINGS

FIG. 1 depicts methods for labeling of total live bacteria in environmental samples using D-amino acid analogs according to some embodiments of the disclosure.

FIG. 2A-E depicts NADA-Green labeling of only live bacteria.

FIG. 3 depicts the time course of labeling live cells with NADA-Green, according to some embodiments of the disclosure.

FIG. 4 depicts the labeling of gram-positive and gram-negative bacteria with NADA-Green, according to some embodiments of the disclosure.

FIGS. 5A-B depict the quantification of labeled cells. FIG. 5A depicts the quantification of cells labeled with NADA-Green, according to some embodiments of the disclosure. FIG. 5B depicts the quantification of cell labelling efficiency by NADA-Green, according to some embodiments of the disclosure.

FIG. 6 illustrates a scheme for metabolic labeling followed by bioorthogonal chemical detection, according to some embodiments of the disclosure. Figure taken from Siegrist et al., 2015, FEMS Microbiology Reviews,

FIG. 7 depicts both components of a two-step click reaction are required for cell labeling, according to some embodiments of the disclosure.

FIG. 8 shows a time course of incubation with EDA in different gram-negative and -positive bacteria, according to some embodiments of the disclosure.

FIG. 9 depicts time course of incubation with EDA in different gram-negative and -positive bacteria on an ferrofluidic assay system, according to some embodiments of the disclosure.

FIG. 10 depicts two-step labeling process using AZDye633 picolyl azide (Click Chemistry Tools, Scottsdale, AZ) on PIPER (A-B) and Fluorescence Microscopy (C), according to some embodiments of the disclosure.

FIGS. 11A-B depict two-step labeling using the “clickable” D-amino acid, EDA, according to some embodiments of the disclosure.

FIGS. 12A-B depict labeling of mixed culture with NADA-Green, according to some embodiments of the disclosure.

FIGS. 13A-C depict media supplementation with metabolism boosters improves labeling of cold-stressed cells, according to some embodiments of the disclosure.

FIG. 14 depicts the slowing of doubling of fast-growing cells by DL-Serine hydroxamate, without preventing labeling, according to some embodiments of the disclosure.

FIGS. 15A-B depict an overview of an ferrofluidic assay platform used to perform methods according to some embodiments of the disclosure.

FIG. 16 depicts an image recognition algorithm for identifying targets for an assay system according to some embodiments of the disclosure.

FIG. 17A shows a schematic of an assay workflow according to some embodiments of the disclosure.

FIG. 17B is a perspective view of an assay cartridge for use in a ferrofluidic assay device/platform. according to some embodiments of the disclosure.

FIG. 18 depicts methods for labeling of total live bacteria in environmental samples using D-amino acid analogs according to some embodiments of the disclosure.

FIG. 19 depicts the slowing of doubling of fast-growing cells by chloramphenicol, without preventing labeling, according to some embodiments of the disclosure.

FIGS. 20A-20D depicts three-step labeling using EDA in combination with dipeptide EDA-DA, according to some embodiments of the disclosure.

FIGS. 21A-21C depicts the results of introduction of an ethanol treatment step prior to click labeling, according to some embodiments of the disclosure.

FIGS. 22A-22B depicts metabolic labeling of live bacteria using strain promoted alkyne-azide cycloaddition (SPAAC) as an alternative to copper-catalyzed azide-alkyne cycloaddition (CuAAC), according to some embodiments of the disclosure.

FIGS. 23A-23C depicts that the metabolic labeling method detects live bacteria spiked into poultry carcass rinses, according to some embodiments of the disclosure.

FIGS. 24A-24C depicts the metabolic labeling method can detect innate live bacterial populations in poultry facility swab samples, according to some embodiments of the disclosure.

FIGS. 25A-25C depicts the metabolic labeling method can detect innate live bacterial populations in poultry carcass rinse samples, according to some embodiments of the disclosure.

FIGS. 26A-26B depicts that enumeration of live bacteria by the metabolic labeling method in reconstituted swab samples shows good correlation to the “gold standard” plate count method, according to some embodiments of the disclosure.

FIG. 27 depicts fluorescence microscopy images of bacterial species listed in Table 1 labeled using the 3-step labeling protocol.

### DETAILED DESCRIPTION AND EXAMPLES

The present disclosure provides methods, systems, and devices for the in-situ labeling of complex environmental samples, specifically mixed populations of total live bacteria, using fluorescent D-amino acids or “clickable” D-amino acids (which can be labeled with fluorophores that have been modified to be click chemistry (i.e., bioorthogonal) fluorophores. The disclosure further provides embodiments for quantifying the TVB by cell manipulation using, for example, ferrofluid within a microfluidic device (e.g., Ancera Inc.'s Piper™ system/platform including cartridge device).

In some embodiments, the present disclosure is directed to a process of labeling a mixed population of bacterial cells in environmental samples using D-amino acid analogs with automated sample processing for visualization, image collection, recognition of individual bacterial cells in those images, and their quantitation.

In some embodiments, the methods of the present disclosure comprise detecting and quantifying live bacteria in environmental swab samples.

In some embodiments, a system is provided which is configured to quantify one or more fluorescently labeled live bacteria from an environmental sample, including a ferrofluid-based assay composed of a sample or plurality of samples mixed with ferrofluid, a microfluidic cartridge containing a plurality of windows, an imager configured to image each window of the cartridge either separately or together, an instrument which controls the flow of samples through the cartridge and allows non-contact cell manipulation via a magnetic field generated by a printed circuit board (PCB), and assay processing components comprising at least one of reagents and controls. The system is configured to at least one of move or otherwise locate the labeled bacteria to one or more of the windows where they can be visualized and/or imaged, and then quantified.

In some embodiments, the live bacteria contained within the sample are allowed to react with a fluorophore-modified D-amino acid which is incorporated in the peptidoglycan (PG) layer of the bacterial cell wall.

In some embodiments, the live bacteria contained within the sample are allowed to react with a clickable bioorthogonal-tag-modified D-amino acid which is incorporated in the peptidoglycan layer of the bacterial cell wall. In some embodiments, the bacteria comprising the clickable bioorthogonal-tag is further modified by covalent attachment of a fluorescent tag using click chemistry.

In some embodiments, the methods of the present disclosure detect and quantify live bacteria within the ranges of 102 to 107 colony forming units (CFU)/swab or higher.

In some embodiments, the methods of the present disclosure detect and quantify live bacteria in 3 hours or less.

In some embodiments, the methods of the present disclosure are useful for plant operations and Food Safety Quality Assurance (FSQA) teams at poultry processing plants.

In some embodiments, the methods of the present disclosure are useful in optimizing microbial control processes and evaluating the effectiveness of anti-microbial interventions.

Without wishing to be bound to any theory, the methods of the present disclosure are useful for providing total live bacteria information for real-time interventions, whereas existing methods rely on cell growth or colony formation and take 24-48 hours.

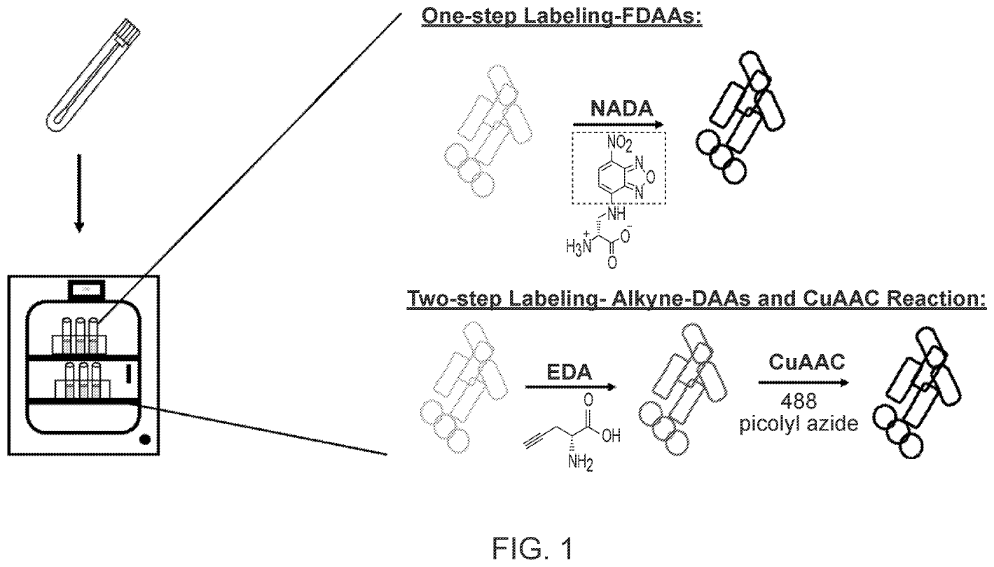

To enable a total live bacteria assay without culturing the sample, some embodiments of the present disclosure use metabolic labeling of bacterial cell walls with D-amino acid analogs (see e.g., FIG. 1). D-amino acids are uniquely used by bacteria to synthesize peptidoglycan, which is a cell wall structure found only in bacteria. Some D-amino acids are used by many bacteria, whereas others are highly specialized. D-alanine is found in peptidoglycan of all bacteria, making it particularly useful for this disclosure. The peptidoglycan biosynthetic pathway in bacteria has inherent promiscuity which allows it to incorporate small molecules conjugated to a D-amino acid backbone at sites of new peptidoglycan synthesis. As such, D-amino acids analogs modified with fluorophores or a bioorthogonal tag (e.g. ethynyl-D-alanine [EDA], azido-D-alanine [ADA], or a dipeptide such as ethynyl-D-alanyl-D-alanine [EDA-DA]) can be incorporated into the existing peptidoglycan of taxonomically diverse bacterial species in real time. Since peptidoglycan synthesis is only possible in actively growing organisms, only live cells can incorporate these artificial D-amino acids in a microscopically detectable amount, making them an ideal trace for a TVB assay.

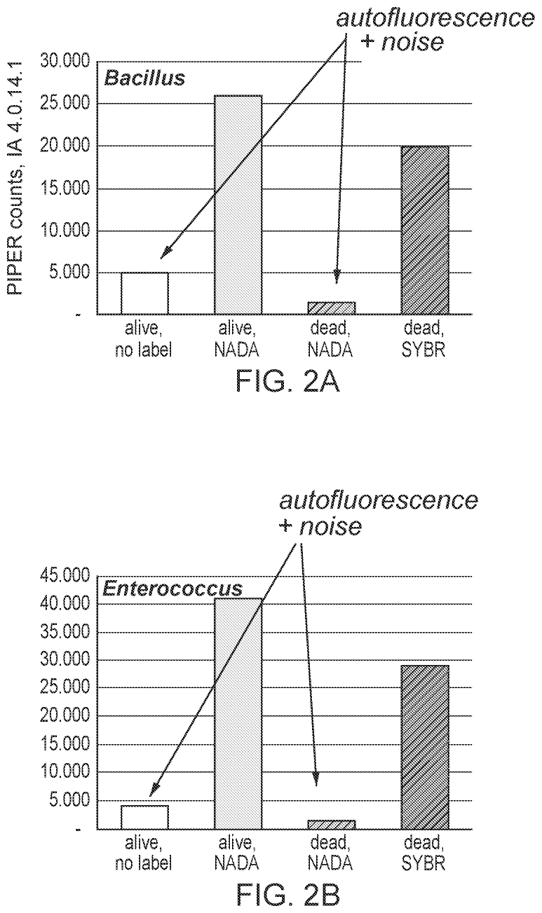

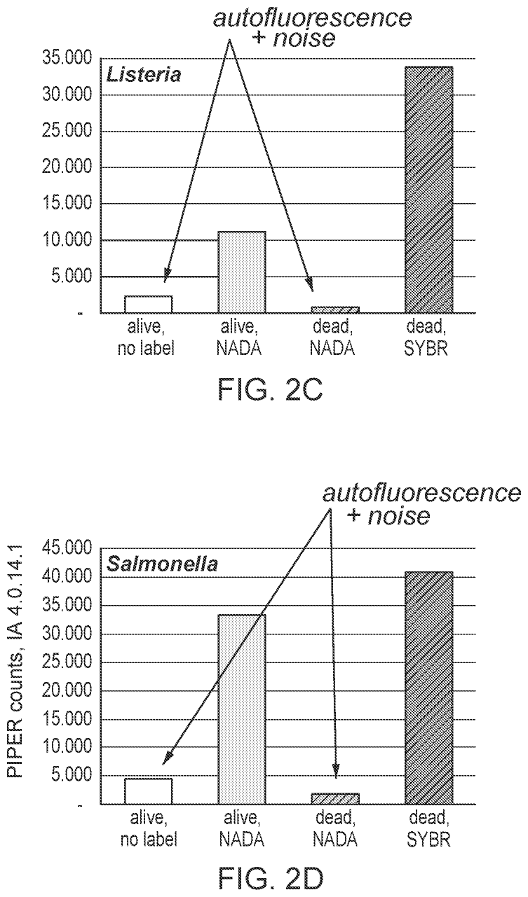

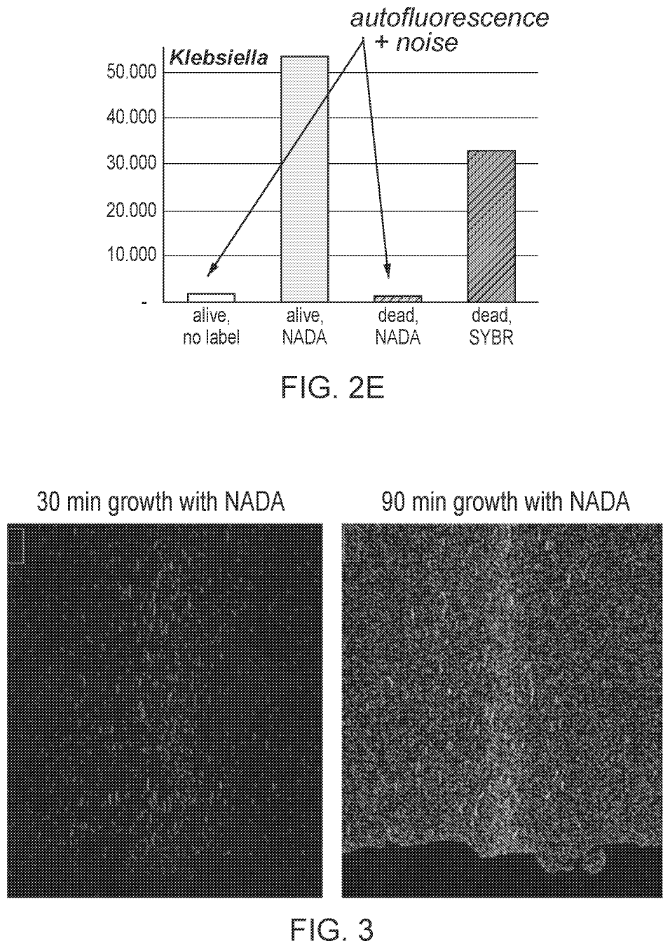

Accordingly, as shown in FIG. 2A-E, NADA-Green labels live bacteria and illustrates five (5) different strains (Bacillus, Enterococcus, Listeria, Salmonella, and Klebsiella) of log phase bacteria growing at 37° C. in brain heart infusion (BHI) media or heat-killed for 15 minutes at 70° C. were incubated with or without the fluorescent D-amino acid, NADA-Green at 1 mM (Bio-Techne, Minneapolis, MN) for 60 minutes. Cells were pelleted to remove the excess dye and resuspended in 1X phosphate buffered saline (PBS). Each sample was then split into two tubes. To one of the tubes, SYBR Green (ThermoFisher Scientific, Waltham, Massachusetts) was added in 1:100 dilution. All samples were processed on Ancera Inc.'s PIPER™ platform (“PIPER”) in a non-capture assay and analyzed by a bacterial image recognition algorithm to determine labeled cell counts. Only live cells labeled with NADA-Green were counted above background noise. SYBR labeling confirmed that the dead cells remained intact on the instrument.

Other methods (e.g., Jepras et. al, 1995, Applied and Environmental Microbiology Vol. 61, p. 2696-2701; Davey and Guyot, 2020, Cytometry e72, Volume 93; Flint et al., 2006, International Diary Journal 16:379-384) have been described for live cell staining with fluorescent dyes (e.g. esterase substrates, such as CFDA or FDA, dyes that rely on membrane potential for staining, such as rhodamine 123 or DiBAC₄, or dyes that depend on membrane integrity to enter cells, such as propidium iodide), but typically these methods cannot distinguish bacteria from other live cells, including yeast and mold, in the samples. Unlike generic metabolic labels (e.g. esterase substrates or ATP), which may detect any live cells present in a sample, D-amino acids can only be incorporated in bacterial cell walls and will not detect other microbes, such as yeast or mold spores, that may be present in a sample.

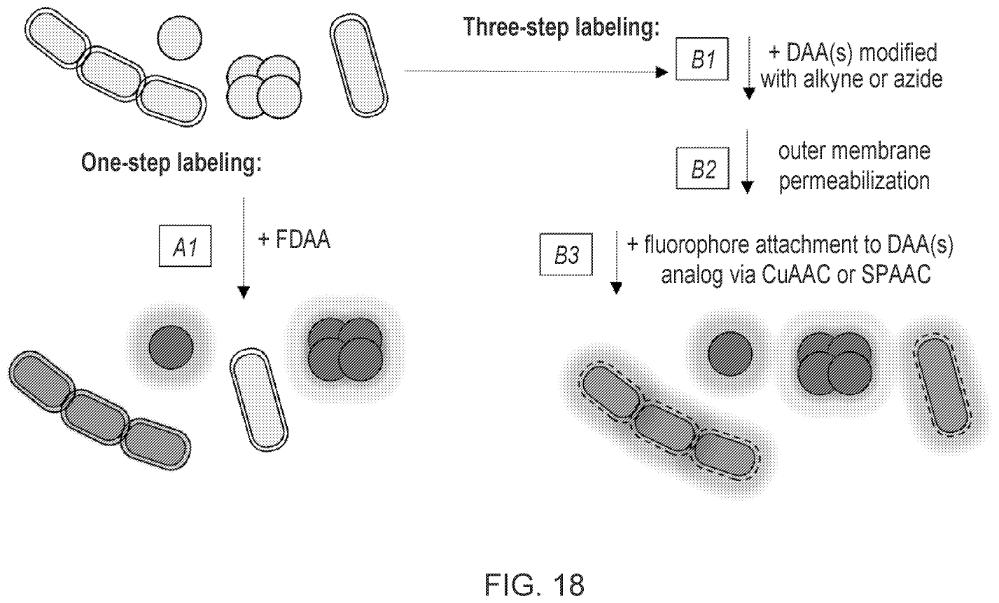

Two approaches were used for introducing the fluorophore into bacterial cell walls through metabolic labeling of peptidoglycan (FIG. 1, 18). As one approach, fluorescent D-amino acids (FDAAs) can be used for single-step labeling of peptidoglycans in bacterial cells by a simple protocol which involves minimal perturbation of the cells (FIGS. 3-5). In the approach depicted in FIG. 18 path A1, fluorescent D-amino acids (FDAAs), such as the D-Ala analog NADA, can be used for single-step labeling of peptidoglycans in bacterial cells by a simple addition during growth which involves minimal perturbation of the cells. However, limited diffusion through the outer membrane of gram-negative bacteria (signified by oval shapes with double lines) results in poor or no labeling of some species. As an alternative, the three-step labeling approach depicted in FIG. 18 path B1-B3 was designed to circumvent this limitation. Here, the sample containing bacteria is first incubated with a D-Ala analog modified with an alkyne or azide group (B1). The small size of such analogs compared to FDAAs allows uptake through the outer membrane of gram-negative bacteria and access to the sites of peptidoglycan synthesis and/or modification. Next, the sample containing bacteria is treated with a permeabilization agent to disrupt the outer membrane barrier of gram-negative bacteria (B2). Finally, a fluorophore molecule modified with an appropriate reactive group can be ligated to the D-Ala analog incorporated into the peptidoglycan at B1 via copper-catalyzed azide-alkyne cycloaddition (CuAAC) or strain promoted alkyne-azide cycloaddition (SPAAC) (B3). Since the outer membrane does not hinder diffusion of the larger fluorophore molecules towards the sites of ligation, the detection of gram-negative bacteria by the three-step method is improved dramatically.

In some embodiments, the present disclosure is directed to path A1 of FIG. 18, path A1 which is depicted in, FIG. 3 which shows the time course of labeling with NADA-Green, showing the log phase Salmonella Typhimuirum growing at 37° C. in BHI media as incubated with NADA-Green at 1 mM (Bio-Techne, Minneapolis, MN) for 30 minutes or 90 minutes. Cells were pelleted by centrifugation (10 min 10,000 g) to remove the excess dye, resuspended in 1×PBS, mixed with ferrofluid, and processed on PIPER using a cartridge having areas coated with a Salmonella-specific antibody. NADA-Green does not interfere with cell growth or capture by the antibody, and labeled bacteria cells can be detected on PIPER.

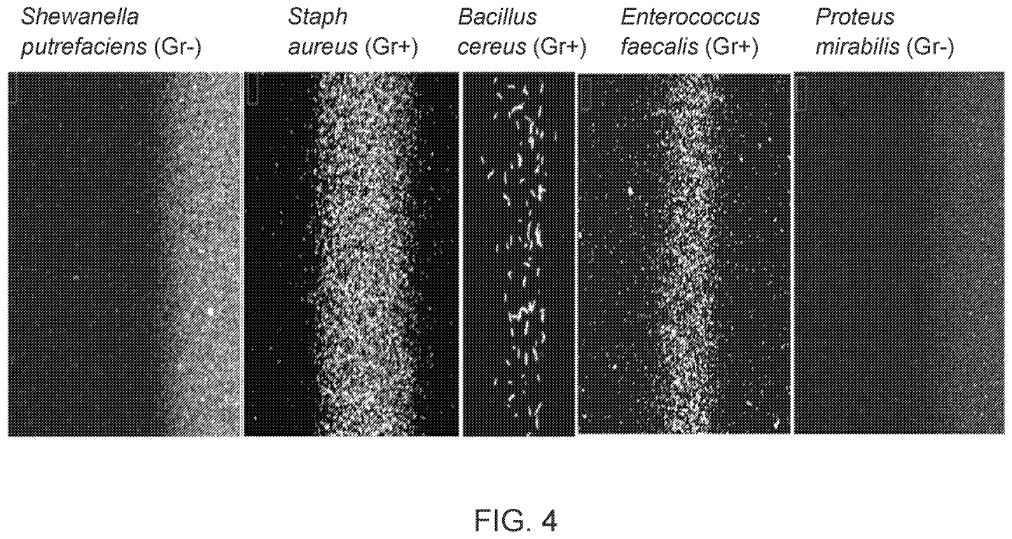

FIG. 4 depicts the labeling of gram-positive and gram-negative bacteria with NADA-Green with the five (5) different strains of log phase bacteria (see FIGS. 2A-E) growing at 37° C. in BHI media, and then incubated with 1 mM NADA-Green (Bio-Techne, Minneapolis, MN) for 90 minutes. Cells were pelleted by centrifugation (10 min 10,000 g) to remove the excess dye, resuspended in 1×PBS, mixed with ferrofluid, and processed on PIPER in a non-capture assay. NADA-Green labels both gram-positive and gram-negative bacteria.

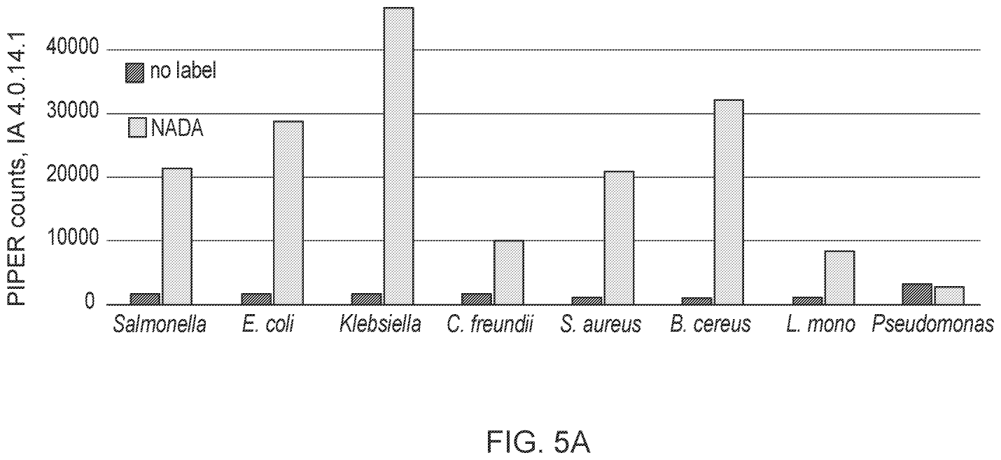

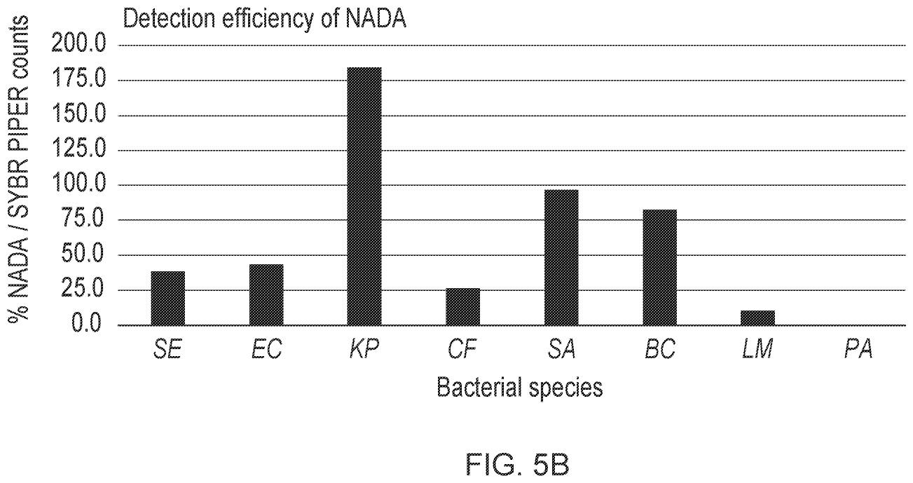

FIG. 5A depicts the quantification of cells labeled with NADA-Green in eight (8) different strains of log phase bacteria. FIG. 5B depicts the quantification of cell labelling efficiency by NADA-Green in eight (8) different strains of log phase bacteria (SE—Salmonella enterica; EC—Escherichia coli; KP—Klebsiella pneumoniae; CF—Citrobacter freundii; SA—Staphylococcus aureus; BC—Bacillus cereus; LM—Listeria monocytogenes; PA—Pseudomonas aeruginosa), determined as a percentage of NADA-labelled PIPER cell counts to the SYBR-labelled PIPER cell counts. Cells growing at 37° C. in BHI media were incubated with 1 mM NADA-Green (Bio-Techne, Minneapolis, MN) for 60 minutes. Cells were pelleted by centrifugation (10 min 10,000 g) to remove the excess dye, resuspended in 1×PBS, mixed with ferrofluid, and processed on PIPER in a non-capture assay. An automated bacterial image recognition algorithm determined labeled cell counts. All labeled strains except Pseudomonas were detected by the image recognition algorithm.

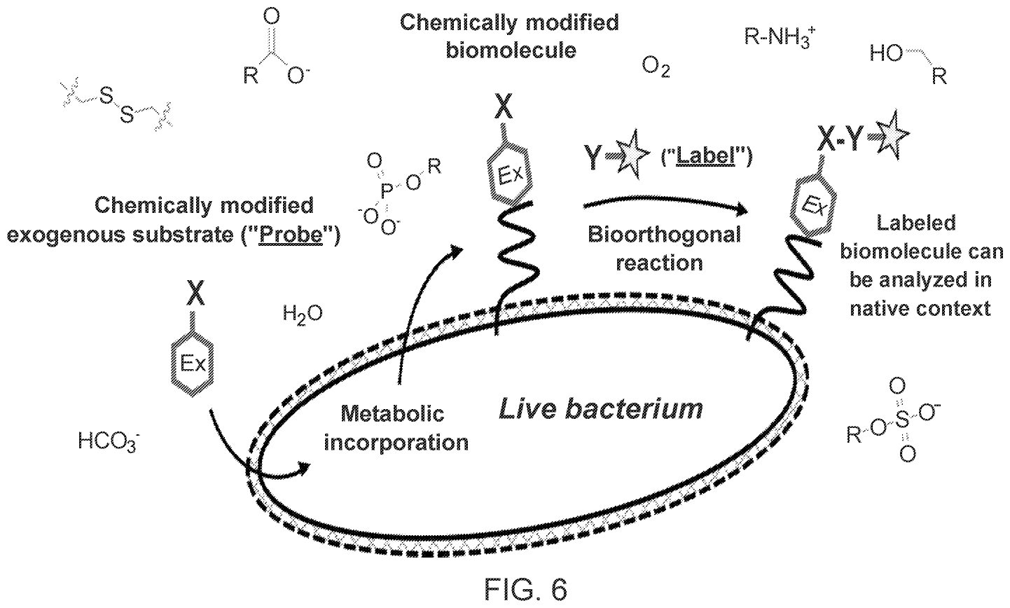

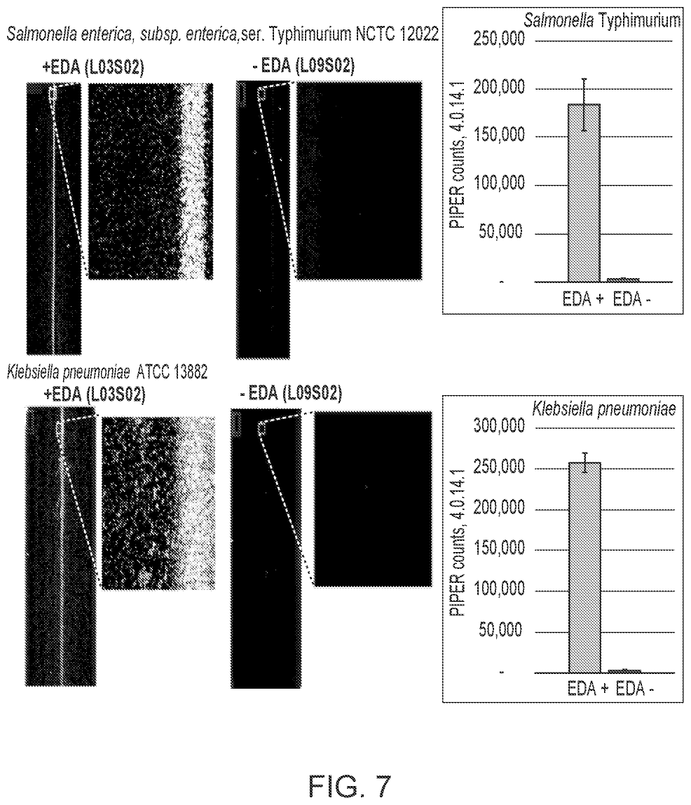

As an alternative approach to FDAAs, peptidoglycan labeling can be achieved in a two-step reaction by use of biorthogonal chemistry (FIG. 6). First, a D-amino acid analog modified with a chemical functional group is added to media containing live bacteria and incubated until the desired level of peptidoglycan decoration is achieved. Then, a fluorescent dye containing a complementary functional group is conjugated to the reactive functional group on the D-amino acid (e.g. copper-assisted click reaction [CuAAC; copper-catalyzed 1,3-dipolar azide-alkyne cycloaddition], copper-free click reaction [SPAAC; strain-promoted alkyne-azide cycloaddition], or other suitable biorthogonal chemistry) (FIGS. 7-10).

FIG. 7 depicts both components of the two-step CLICK reaction are required for cell labeling. Salmonella Typhimurium (top panel) and Klebsiella pneumoniae (bottom panel) were grown overnight at 37° C. The next day, cells were diluted into fresh media and grown until they reached log-phase. The last 30 min of incubation were carried out in the presence or absence of 1 mM of EDA (D-propargylglycine, AK Scientific, Union City, CA). Cells were harvested by centrifugation (10 min 10,000 g), and supernatant was discarded. Bacteria were then washed, fixed by incubation with 4% paraformaldehyde, pelleted by centrifugation (10 min 10,000 g), and supernatant was discarded. Cell pellets were resuspended in click reaction solution (containing 200 μM copper sulfate (II) (MilliporeSigma, Burlington, MA), 128 μM THPTA (Tris-hydroxypropyltriazolylmethylamine) ligand (Lumiprobe, Hunt Valley, MD), 2.5 mM Ascorbic acid (MilliporeSigma, Burlington, MA), 25 μM CF488 picolyl azide fluorescent dye (Biotium, Fremont, CA) and incubated for 30 min at room temperature. Cells were pelleted by centrifugation (10 min 10,000 g), supernatant was discarded, and pellets were resuspended 1 mL of 1× PBS. The wash step was repeated two more times. Finally, pellets were resuspended in PBS, mixed with ferrofluid, and processed on PIPER in a non-capture assay. An automated bacterial image recognition algorithm determined labeled cell counts (graphs). No signal is observed when the click reagents are added in the absence of EDA.

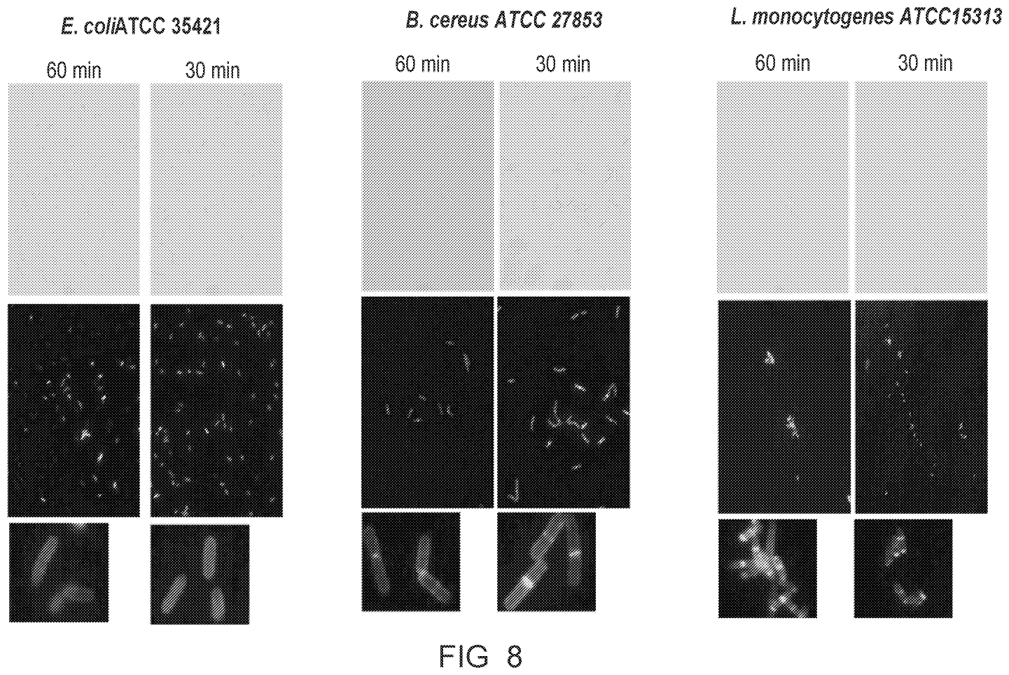

FIG. 8 shows a time course of incubation with EDA in different gram-negative and -positive bacteria. E. coli (A), L monocytogenes (B), and B. cereus (C) were grown overnight at 37° C. The next day, cells were diluted into fresh media and grown until they reached log-phase. The last 30 or 60 min of incubation were carried out in the presence of 1 mM of EDA (D-propargylglycine, AK Scientific, Union City, CA). Cells were harvested by centrifugation (10 min 10,000 g), and supernatant was discarded. Bacteria were washed with 1 mL of 1× PBS, pelleted by centrifugation (10 min 10,000 g), and supernatant was discarded. Cell pellets were resuspended in click reaction solution containing 200 μM copper sulfate (II) (MilliporeSigma, Burlington, MA), 128 μM THPTA (Tris-hydroxypropyltriazolylmethylamine) ligand (Lumiprobe, Hunt Valley, MD), 2.5 mM Ascorbic acid (MilliporeSigma, Burlington, MA), 25 μM CF488 picolyl azide fluorescent dye (Biotium, Fremont, CA) and incubated for 30 min at room temperature. Cells were pelleted by centrifugation (10 min 10,000 g), supernatant was discarded, and pellets were resuspended in 1 mL of 1× PBS. The wash step was repeated two more times. Finally, pellets were resuspended in PBS and analyzed by microscopy.

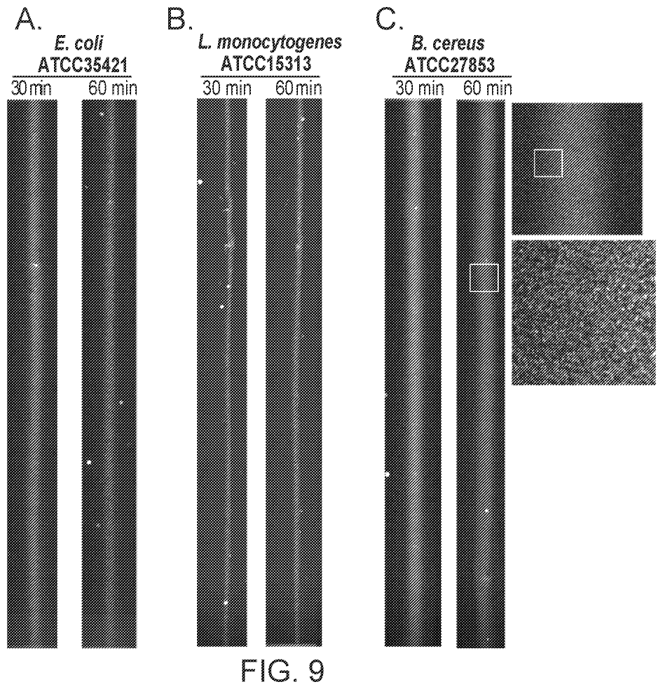

FIG. 9 depicts a time course of incubation with EDA in different gram-negative and -positive bacteria on PIPER. E. coli (A), L monocytogenes (B), and B. cereus (C) were grown overnight at 37° C. The next day, cells were diluted into fresh media and grown until they reached log-phase. The last 30 or 60 min of incubation were carried out in the presence of 1 mM of EDA (D-propargylglycine, AK Scientific, Union City, CA). Cells were harvested by centrifugation (10 min 10,000 g), and supernatant was discarded. Bacteria were washed with 1 mL of 1× PBS, pelleted by centrifugation (10 min 10,000 g), and supernatant was discarded. Cell pellets were resuspended in click reaction solution containing 200 μM copper sulfate (II) (MilliporeSigma, Burlington, MA), 128 μM THPTA ligand (Lumiprobe, Hunt Valley, MD), 2.5 mM Ascorbic acid (MilliporeSigma, Burlington, MA), 25 μM CF488 picolyl azide fluorescent dye (Biotium, Fremont, CA) and incubated for 30 min at room temperature. Cells were pelleted by centrifugation (10 min 10,000 g), supernatant was discarded, and pellets were resuspended in 1 mL of 1× PBS. The wash step was repeated two more times. Finally, pellets were resuspended in PBS, mixed with ferrofluid and processed in the non-capture assay on PIPER.

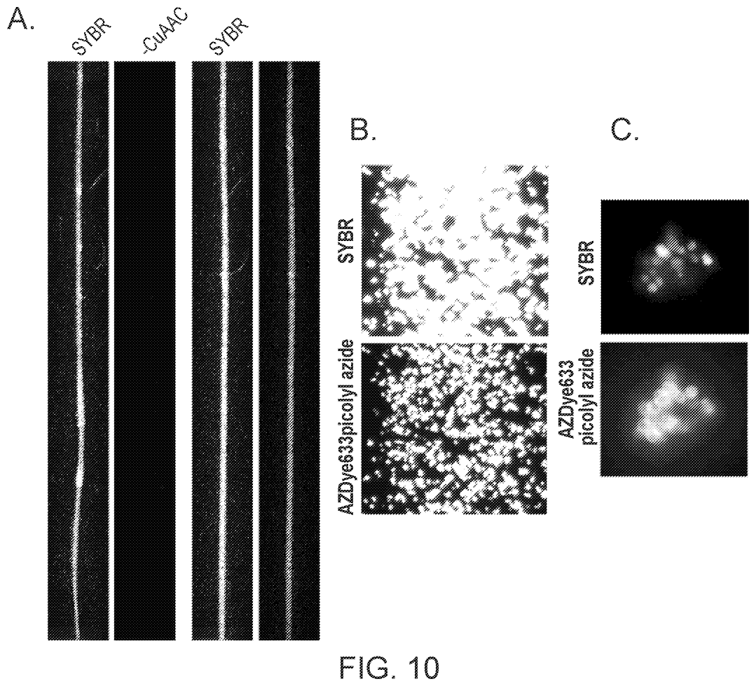

FIG. 10 depicts two-step labeling with AZDye633 picolyl azide (Click Chemistry Tools, Scottsdale, AZ) on PIPER (A-B) and fluorescence microscopy (C). S. auerus was grown overnight at 37° C. The next day, cells were diluted into fresh media and grown until they reached log-phase. The last 30 min of incubation were carried out in the presence of 1 mM of EDA (D-propargylglycine, AK Scientific, Union City, CA). Cells were harvested by centrifugation (10 min 10,000 g), and supernatant was discarded. Bacteria were washed with 1 mL of 1× PBS, pelleted by centrifugation (10 min 10,000 g), and supernatant was discarded. Cell pellets were resuspended in click reaction solution containing 200 μM copper sulfate (II) (MilliporeSigma, Burlington, MA), 128 μM THPTA ligand (Lumiprobe, Hunt Valley, MD), 2.5 mM Ascorbic acid (MilliporeSigma, Burlington, MA), 25 μM AZDye633 picolyl azide fluorescent dye (Click Chemistry Tools, Scottsdale, AZ) and incubated for 30 min at room temperature. Cells were pelleted by centrifugation (10 min 10,000 g), supernatant was discarded, and pellets were resuspended in 1 mL of 1× PBS. SYBR Green (ThermoFisher Scientific, Waltham, Massachusetts) was added in 1:100 dilution to all the samples and the cells were incubated for 5 min. The wash step was repeated two more times. Finally, pellets were resuspended in PBS, mixed with ferrofluid and processed in the non-capture assay on PIPER. Images were collected in both the green and far-red channels, on PIPER and by conventional microscopy. All images were processed using ImageJ.

In some embodiments, the present disclosure is directed to the second approach described in FIG. 1 and path B1-B3 of FIG. 18, where, the chemical groups on the D-amino acid and fluorescent dye are bioorthogonal, meaning that they react efficiently with each other but lack any natural substrate in the living cells. Examples of such D-amino acid analogs include, but are not limited to, “clickable” D-amino acids (e.g. EDA, ADA, EDA-DA). The advantage of the two-step strategy is that the small size of “clickable” D-amino acids compared to FDAAs allows for more efficient labeling of gram-negative bacteria that possesses the outer membrane, acting as a barrier for larger molecules. FDAAs have been synthesized with different spectral properties (Hsu et al. 2017), and numerous “clickable” fluorophores with varied spectral properties are available from commercial sources, expanding the range of fluorophores that can be used for labeling. Either the one-step FDAA labeling method or the two-step “clickable” D-amino acid labeling method can be used in the methods of the present disclosure. While FDAAs and D-amino acid analogs with “clickable” functional groups can label bacteria (see e.g., US2019/0024132 A1, U.S. Pat. Nos. 10,544,444, 10,016,498), none of the disclosures teach or suggest the use of these labeling technologies for evaluating mixed populations of bacteria cells to determine total live bacteria in a sample. The use cases described in the prior art are limited to studying cell wall synthesis in individual bacteria cells or to identifying agents that can inhibit peptidoglycan synthesis.

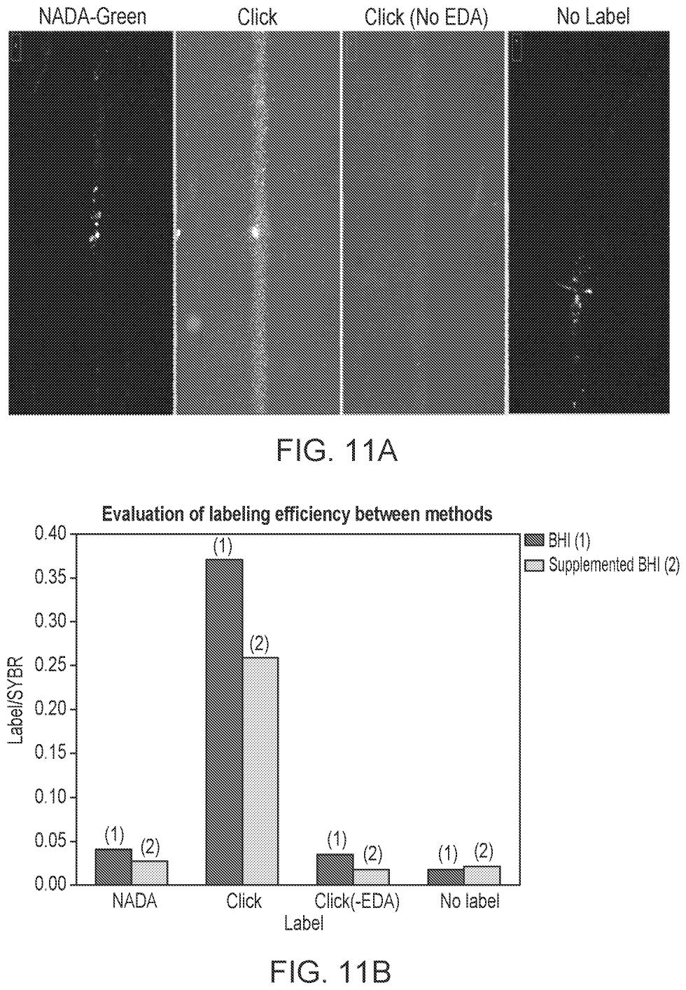

Universal labeling of all bacteria presents some challenges. D-amino acid analogs must first penetrate the outer membrane of gram-negative bacteria to be incorporated into peptidoglycan. The smaller size of the “clickable” D-amino acids compared to the FDAAs helps achieve higher labeling efficiency for some gram-negative bacteria. Accordingly, and for example, FIGS. 11A-B depict two-step labeling using the “clickable” D-amino acid, EDA, which improves detection of Pseudomonas. Log phase Pseudomonas growing at 37° C. in BHI (with [Supplemented BHI, red bars] or without [blue bars] supplementation with 50 mM glucose and 10 mM sodium pyruvate) was incubated for 60-90 minutes with 1 mM NADA-Green (Bio-Techne, Minneapolis, MN); or for 30 minutes in the presence or absence of 1 mM clickable D-amino acid, EDA (D-propargylglycine, AK Scientific, Union City, CA). For the unlabeled cells or cells labeled with NADA-Green, the cells were pelleted after incubation and resuspended in 1×PBS. For the cells grown in the presence or absence of EDA, the cells were pelleted after incubation by centrifugation (10 min 10,000 g), and supernatant was discarded. Bacteria were washed with 1 mL of 1× PBS, pelleted by centrifugation again (10 min 10,000 g), and supernatant was discarded. Cell pellets were resuspended in click reaction solution containing 200 μM copper sulfate (II) (MilliporeSigma, Burlington, MA), 128 μM THPTA ligand (Lumiprobe, Hunt Valley, MD), 2.5 mM Ascorbic acid (MilliporeSigma, Burlington, MA), 25 μM CF488 picolyl azide fluorescent dye (Biotium, Fremont, CA) and incubated for 30 min at room temperature. Cells were pelleted by centrifugation (10 min 10,000 g), supernatant was discarded, and pellets were resuspended in 1 mL of 1× PBS. SYBR Green (ThermoFisher Scientific, Waltham, Massachusetts) was added in 1:100 dilution to all the samples and the cells were incubated for 5 min. The wash step was repeated two more times. Finally, pellets were resuspended in PBS, mixed with ferrofluid and processed in the non-capture assay on PIPER. FIG. 11A shows PIPER images of Pseudomonas labeled with NADA-Green (“NADA”), complete 2-step click labeling (“Click”), control for the specificity of 2-step labeling lacking EDA (“Click (-EDA)”), or no label. FIG. 11B shows cell counts by the image recognition algorithm for each of the labeling methods relative to counts obtained by SYBR labeling. Highest detection efficiency was observed using EDA+Click reaction solution.

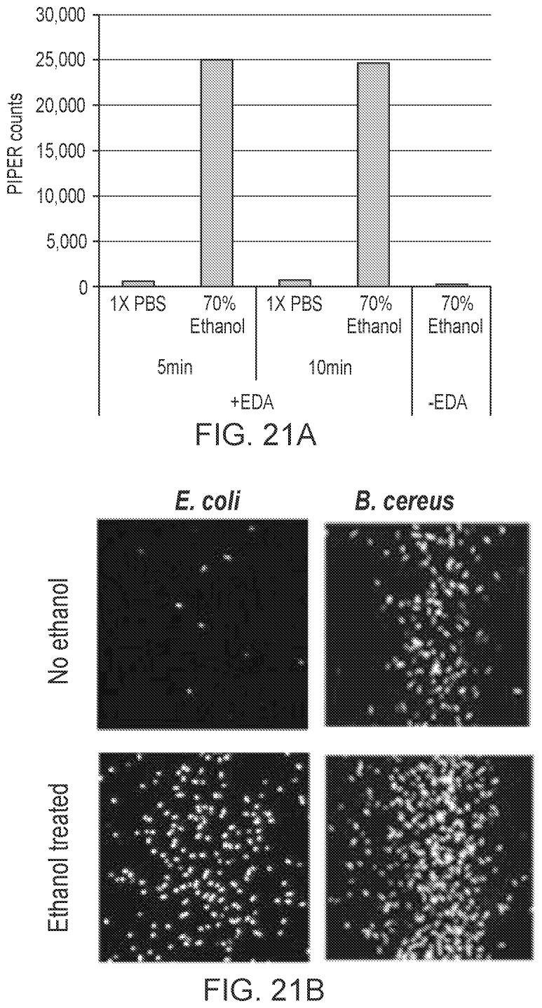

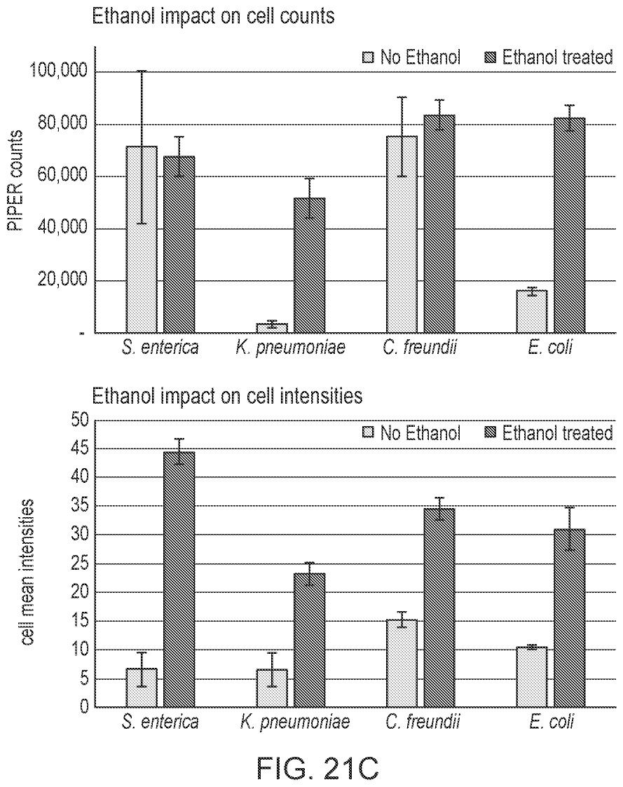

A fixation step prior to the click reaction, or addition of a chemical agent during the click reaction, can be used in some embodiments to prevent the loss of signal due to fast peptidoglycan turnover, to improve conjugation efficiency by disrupting the outer membrane of Gram-negative bacteria and exposing the peptidoglycan (e.g., see FIG. 7 and FIG. 21), and/or to reduce non-specific staining of the sample components by the fluorescent dye. Such agents include, but are not limited to, organic solvents (ethanol, isopropanol, DMSO), 4% paraformaldehyde, or mild detergents (saponin, Triton-X, Tween, sodium dodecyl sulfate).

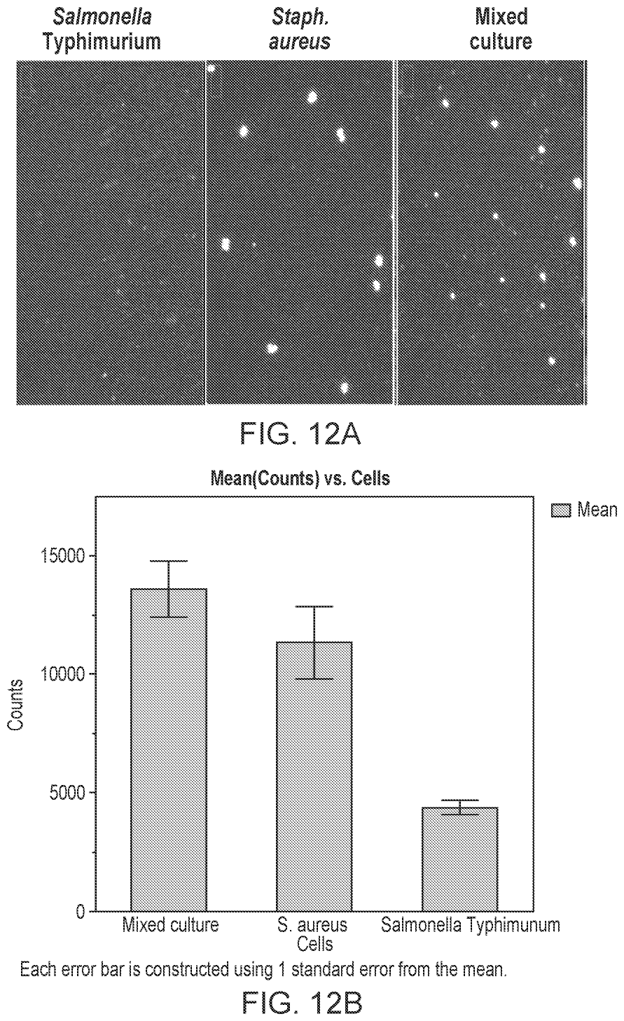

Even with efficient uptake of the D-amino acid analog, gram-negative bacteria may not stain as brightly as gram-positive bacteria because gram-negative bacteria have a much thinner peptidoglycan layer. Examples herein show that a mixture of gram-negative and gram-positive bacteria can be labeled and detected in the PIPER platform despite differences in signal intensities. For example, FIGS. 12A-B depict labeling of a mixed culture with NADA-Green. A log phase culture of Salmonella Typhimurium, Staphylococcus aureus, or a mixture of the two strains was incubated at 37° C. with 1 mM NADA-Green Bio-Techne, Minneapolis, MN) for 60 minutes. Cells were pelleted by centrifugation (10 min 10,000 g) to remove the excess dye, resuspended in 1×PBS, mixed with ferrofluid, and processed on PIPER in a non-capture assay. FIG. 12A shows a zoomed in view of a PIPER image for each sample. Salmonella cells appear smaller and dimmer in the image than Staph. aureus cells. FIG. 12B shows plots of PIPER counting data from the image recognition algorithm. Results show that labeling can be done in a mixed culture and counts in the mixed culture are approximately additive of the counts for each individual culture.

More than one precursor of peptidoglycan modified with the same clickable bioorthogonal tag can be used in situations where using only the D-alanine analog does not provide a sufficient degree of peptidoglycan decoration (for example, use of D-lysine or D-isoglutamate analogs in addition to D-alanine analog). In situations where a target bacteria or group of bacteria are characterized by the presence of a unique peptidoglycan precursor, its analog(s) can be used in lieu of the universal D-alanine analog for subsequent click-chemistry labeling. Examples include D-lactate (present only in vancomycin-resistant bacteria) or D-aspartate (present in Lactococcus and Enterococcus). In some embodiments, in addition to the D-amino acid analogs, a lipopolysaccharide (LPS) chemical reporter with a bioorthogonal tag (such as described by Nilsson I., et al. 2017, and Liu et al., 2021) is added to improve the signal for gram-negative bacteria or to discriminate gram-negative from gram-positive bacteria in the sample.

In some embodiments, labeling of the live bacteria sample may utilize: EDA, a small clickable D-amino acid; HADA, a fluorescent D-amino acid; EDA-DA; DA-EDA, two clickable small DAADs.

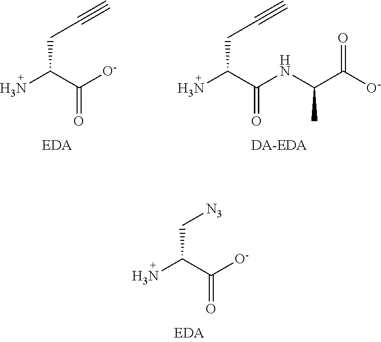

In some embodiments, more than one precursor of peptidoglycan modified with the same clickable bioorthogonal tag can be used. In some embodiments, a combination of EDA with dipeptide EDA-DA can be used. In some embodiments, a combination of EDA and EDA-DA is used at a ratio of about 1:2, about 1:1, or about 2:1. In some embodiments, a combination of EDA and EDA-DA is used at a ratio of about 1:1.

FDAAs include but are not limited to HADA, and NADA.

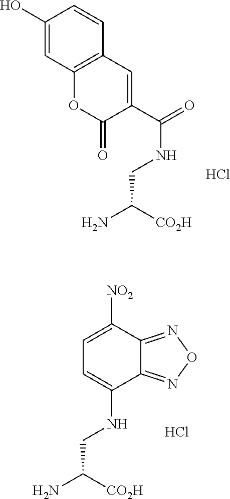 [SMILES: N[C@H](CNC(=O)c1cc2ccc(O)cc2oc1=O)C(=O)O ; Cl ; N[C@H](CNc1ccc([N+](=O)[O-])c2nonc12)C(=O)O ; Cl]

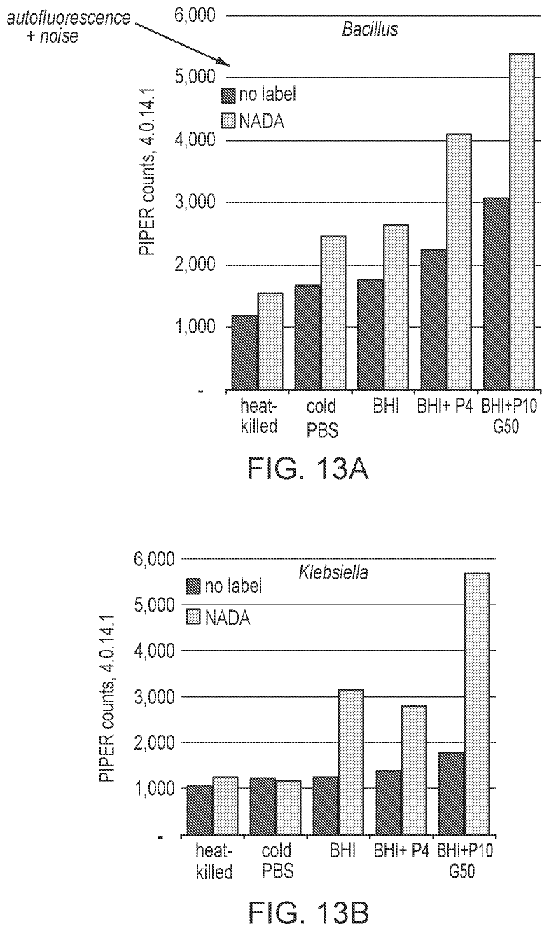

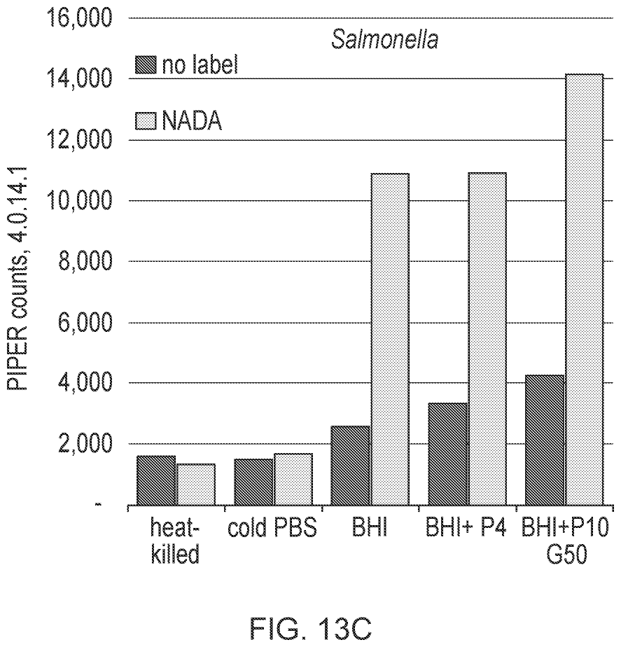

Another challenge for universal labeling is that different bacteria grow at different rates. In order for the assay to remain quantitative, the bacterial population cannot be skewed by doubling of fast-growing bacteria during the metabolic labeling and sample processing steps. To address this issue, small molecules that stimulate the metabolic activity of live cells while controlling for cell doubling can be used in the methods of the present disclosure. Metabolite boosters can include but are not limited to D-(+)-glucose and/or sodium pyruvate (see, e.g., WO2022015845 which is incorporated by reference in its entirety). For example, FIGS. 13A-C depict media supplementation with metabolism boosters improves labeling of cold-stressed cells. A loopful of cells from a 7-day old plate of each of 3 species of bacteria stored at 4° C. was resuspended in 1×PBS. The cells were then either diluted further in cold PBS, diluted in BHI, or diluted in BHI supplemented with 4 mM sodium pyruvate (P4) or 10 mM sodium pyruvate (P10) and 50 mM glucose (G50) and incubated at 37° C. for 1 hour in the presence or absence of 1 mM NADA green (Bio-Techne, Minneapolis, MN) for 60 minutes. Cells were pelleted by centrifugation (10 min 10,000 g) to remove the excess dye, resuspended in 1×PBS, mixed with ferrofluid, and processed on PIPER in a non-capture assay. For two of the three strains (Bacillus cereus and Klebsiella pneumoniae), cell counts were highest when the media was supplemented with both glucose and sodium pyruvate.

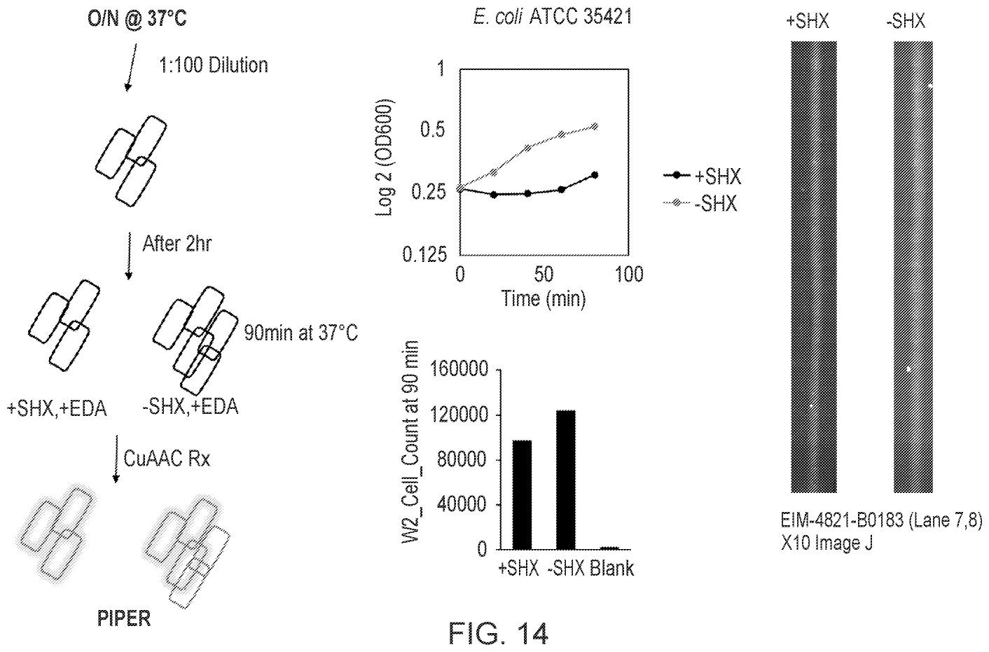

Cell doubling control can be achieved using bacteriostatic agents that do not interfere with peptidoglycan formation, such as, but not limited to, for example chloramphenicol as well as the stringent response inducer, DL-serine hydroxamate (Ferullo et al., 2009). FIG. 14 depicts that DL-Serine hydroxamate slows doubling of fast-growing cells but does not prevent labeling. E. coli was grown overnight at 37° C. The next day, cells were diluted into fresh media and grown for 2 hours. Next, +/−DL-serine hydroxamate (SHX; 1 mg/ml) and EDA (1 mM) were added. OD600 was taken every 20 min for 90 min. Aliquots of cells at the last time point were pelleted by centrifugation (10 min 10,000 g), and supernatant was discarded. Bacteria were washed with 1 mL of 1× PBS, pelleted by centrifugation (10 min 10,000 g), and supernatant was discarded. Cell pellets were resuspended in click reaction solution containing 200 μM copper sulfate (II) (MilliporeSigma, Burlington, MA), 128 μM THPTA ligand (Lumiprobe, Hunt Valley, MD), 2.5 mM Ascorbic acid (MilliporeSigma, Burlington, MA), 25 μM CF488 picolyl azide fluorescent dye (Biotium, Fremont, CA) and incubated for 30 min at room temperature. Cells were pelleted by centrifugation (10 min 10,000 g), supernatant was discarded, and pellets were resuspended 1 mL of 1× PBS. Finally, pellets were resuspended in PBS, mixed with ferrofluid and processed in the non-capture assay on PIPER. An automated bacterial image recognition algorithm determined labeled cell counts.

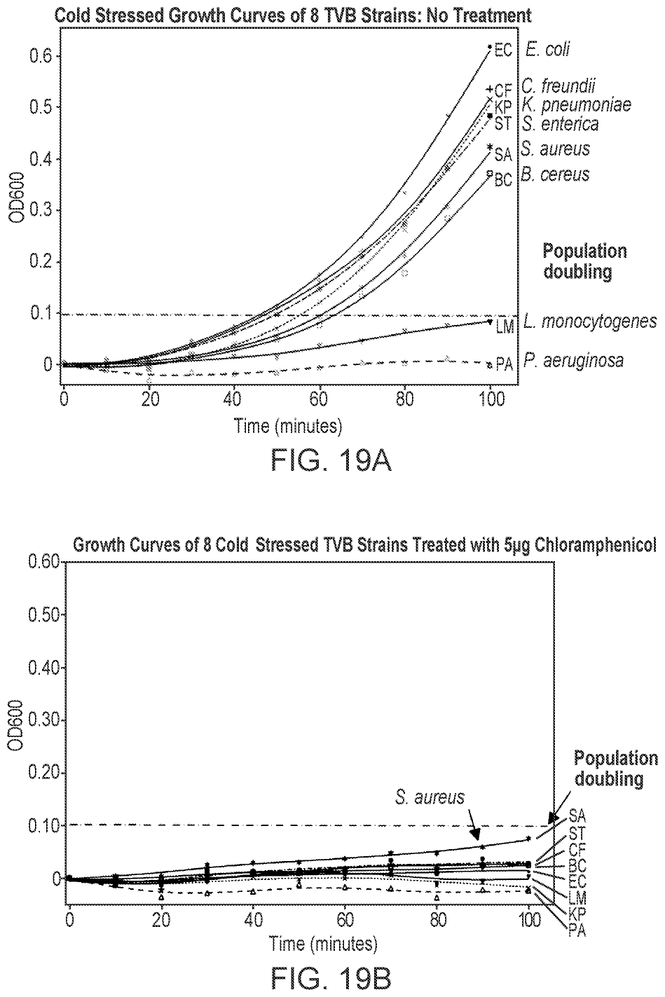

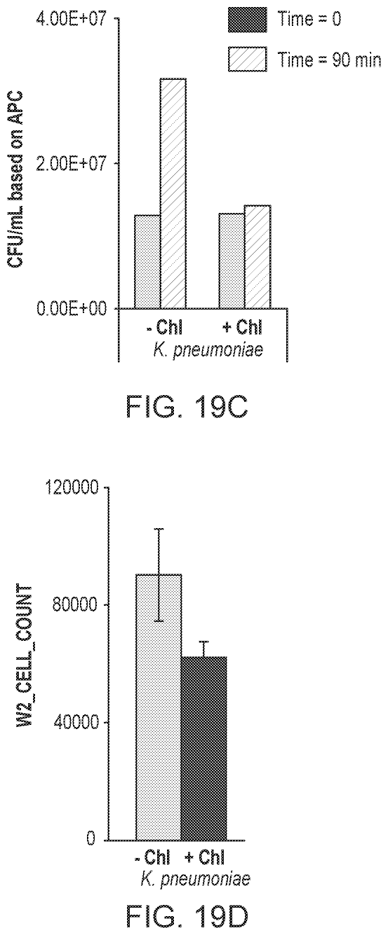

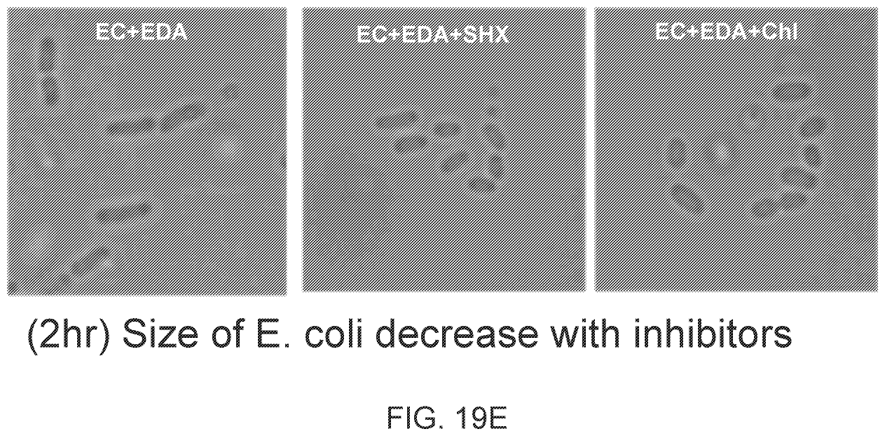

FIG. 19 depicts that chloramphenicol slows doubling of fast-growing cells but does not prevent metabolic labeling. Eight (8) different species of bacteria (Salmonella enterica, E. coli, Klebsiella pneumoniae, Citrobacter freundii, Staphylococcus aureus, Bacillus cereus, Listeria monocytogenes and Pseudomonas aeruginosa) were propagated from frozen stocks onto TSA plates and incubated at 37° C. for 24 hours. To induce cold-stress, a loopful of cells from each plate was transferred into 3 mL of Butterfield's Phosphate Buffer, vortexed to suspend the cells, and placed at 4° C. for 1 day. The following day, cells from each culture were pelleted for 10 minutes at 10,000 g and resuspended in BHI (FIG. 19A) or BHI containing 5 μg/mL of chloramphenicol (FIG. 19B). Each culture was normalized to an absorbance value at 600 nm (OD600) of 0.05 and loaded in triplicate onto a sterile microplate at 0.2 mL per well. The microplate was covered with BreatheEasy film and bacterial growth was monitored using a SpectroStar Nano spectrophotometer (BMG Labtech, Cary, NC) for 24 hours. During growth, the temperature was maintained at 37° C. and the plate was automatically shaken prior to OD600 readings every 10 minutes. Addition of chloramphenicol prevented doubling in all 8 strains within the 2-hour observation window (FIG. 19B), while most strains grew vigorously in the control group (FIG. 19A). OD600 observations were validated using the “gold-standard” petrifilm counts (3M) (FIG. 19C). A cold-stressed Klebsiella pneumoniae (KP) culture prepared as described above was transferred into BHI or BHI containing 5 μg/mL of chloramphenicol and either plated immediately or placed at 37° C. for 90 minutes. Serial dilution and plating on APC petrifilm (3M) was carried out to enumerate cells before and after incubation in each treatment group. Comparison of cell counts confirmed that addition of 5 μg/mL of chloramphenicol prevented cell doubling, while in the control sample (not treated with chloramphenicol) a significant increase in cells was observed. Most importantly, addition of 5 μg/mL of chloramphenicol did not interfere with 3-step metabolic labeling and detection on PIPER (FIG. 19D). A cold-stressed KP culture prepared as described above was transferred into BHI containing 1 mM EDA with or without 5 μg/mL of chloramphenicol, and incubated at 37° C. for 90 min. Cells were harvested by centrifugation (10 min 10,000 g), and supernatant containing media and excess (unincorporated) EDA was discarded. The 2^{nd} step was carried out by resuspending the cells in 0.75 mL of 1×PBS and mixing them with 0.25 mL of 96% ethanol. After a 5 minute incubation at room temperature, cells were pelleted by centrifugation (10 min 10,000 g) and supernatant containing media with ethanol and residual EDA was discarded. The 3^{rd} step was carried out by resuspending cell pellets in 0.25 mL of 1×PBS solution containing 200 μM copper sulfate (II) (MilliporeSigma, Burlington, MA), 400 μM THPTA ligand (Lumiprobe, Hunt Valley, MD), 2.5 mM Ascorbic acid (MilliporeSigma, Burlington, MA), 10 μM CF488 picolyl azide fluorescent dye (Biotium, Fremont, CA), and 5% DMSO (Thermo Fisher Scientific, Waltham, MA). After 30 at room temperature, 1 mL of 1×PBS was added to each reaction, and cells were harvested by centrifugation (10 min 10,000 g). Supernatant containing excess dye and reaction components was discarded, cells were washed with 1 mL of 1×PBS, and cells were pelleted again by centrifugation. Finally, each cell pellet was resuspended in 0.45 mL of 1×PBS and mixed with an equal volume of ferrofluid. All samples were run on PIPER in triplicate and cell counts were generated by image analysis using an automated bacterial image recognition algorithm. A slight decrease in cell counts observed in the sample treated with chloramphenicol in FIG. 19D might be due to changes in cell morphology in response to the antibiotic. FIG. 19E shows dark-field microscopy of E. coli cells grown for 2 hours at 37° C. in BHI with 1 mM EDA in the presence or absence of bacteriostatic agents (SHX-DL-serine hydroxamate, 1 mg/ml; Chl—chloramphenicol, 5 μg/mL).

### Bacteria

In some embodiments, the methods herein are useful in evaluating any live bacteria that would be detectable by total aerobic plate count methods. In some embodiments, the methods herein are useful in evaluating total live bacteria, wherein the bacteria is either gram negative or gram positive or a mixture of both gram negative and gram positive bacteria.

In some embodiments, the bacteria to be analyzed/quantified is gram negative. In some embodiments, the bacteria is gram negative and comprises Aeromonas hydrophila, Bukholderia cenocepacia, Campylobacter jejuni, Citrobacter freundii, Enterobacter sakasakii (Cronobacter), Escherichia coli, Flavobacterium Sp., Hafnia alvei, Klebsiella pneumoniae, Kluyvera Sp., Moraxella catarrhalis, Proteus mirabilis, Pseudomonas aeruginosa, Salmonella Typhimurium, Serratia liquefaciens, Shewanella putrefaciens, Shigella flexneri, Vibrio parahaemolyticus, or Yersinia enterocolitica.

In some embodiments, the bacteria to be analyzed/quantified is gram positive. In some embodiments, the bacteria is gram positive and comprises Bacillus cereus, Bacteroides fragilis, Brochothrix thermosphacta, Clostridium perfringens, Corynebacterium Sp, Enterococcus faecalis, Lactobacillus brevis, Lactococcus lactis, Leuconostoc lactis, Listeria monocytogenes, Staphylococcus aureus, Streptococcus pyogenes, Weissella viridescens.

In some embodiments, the bacteria to be analyzed/quantified is an obligate intracellular pathogen or a facultative intracellular pathogen. In some embodiments, the bacteria to be analyzed/quantified is a Mycobacterium species. Examples of species of Mycobacterium include, but are not limited to, M. tuberculosis, M. bovis, M. bovis strain Bacillus calmette-guerin (BCG) including BCG substrains, M. avium, M. intracellulare, M. africanunum, M. kansasii, M. marinum, M. ulcerans and M. paratuberculosis. Examples of other obligate and facultative intracellular bacterial species include, but are not limited to, Legionella pneumophila, other Legionella species, Salmonella Typhi, other Salmonella species, Shigella species, Listeria monocytogenes, Staphylococcus aureus, Staphylococcus epidermidis, Bacteroides fragilis, other Bacteroides species, Chlamydia pneumoniae, Chlamydia trachomatis, Chlamydia psittaci, Coxiella burnetii, other Rickettsial species, and Ehrlichia species.

In some embodiments, the bacteria to be analyzed/quantified is Francisella tularensis; Listeria monocytogenes; Salmonella; Brucella; Legionella pneumophila; Mycobacterium (e.g., M. tuberculosis, M. leprae, M. bovis, M. avium, M. abscessus); Nocardia (e.g., N. asteroids, N. farcinica, N. nova, N. transvalensis, N. brasiliensis, N. pseudobrasiliensis); Rhodococcus equui; Yersinia pestis; Neisseria (e.g., N. meningitidis, N. gonorrhoeae); Shigella (e.g., S. dysenteriae, S. flexneri, S. boydii, and S. sonnei); Chlamydia (C. trachomatis, C. pneumoniae, C. psittaci); Rickettsia; and Coxsiella. In some embodiments, the bacteria to be analyzed/quantified is Vibrio cholerae, Pseudomonas aeruginosa, and pathogenic Escherichia coli; model organisms such as Escherichia coli, Bacillus subtilis, and Caulobacter cresentus; facultative pathogens such as Streptococcal and Clostridial species; and commensals such as Bacteroides thetaiotamicron. In some embodiments, the bacteria to be analyzed/quantified is Acinetobacter geminorum, Acinetobacter haemolyticus, Acinetobacter lwoffii, Acinetobacter pittii or A. calcoaceticus, Bacillus licheniformis, Bacillus mojavensis, Brachybacterium paraconglomeratum, Chryseobacterium gambrini, Corynebacterium ammoniagenes, Coryneobacterium callunae, Cytobacillus solani, Empedobacter falsenii, Exiguobacterium indicum, Heyndrickxia oleronia, Kocuria tytonicola or K. tytonis, Kurthia gibsonii, Kurthia populi, Macrococcus caseolyticus, Mammaliicoccus lentus, Microbacterium invictum, Microbacterium maritypicum, Microbacterium testaceum, Pseudomonas mosselii, Pseudoxanthomonas mexicana, Rothia nasimurium, Staphylococcus pseudoxylosus, Staphylococcus lloydii or S. kloosii, Staphylococcus nepalensis, Staphylococcus ureilyticus, or Streptococcus pluranimalium.

In some embodiments, small molecules that stimulate the metabolic activity of live cells are added to the samples to improve labeling efficiency. In some embodiments, D-(+)-glucose and/or sodium pyruvate are added to the samples.

In some embodiments, the growth of faster growing bacteria is slowed to prevent disrupting the quantification of the total live bacteria. In some embodiments, bacteriostatic agents that do not interfere with peptidoglycan formation are added to the sample to prevent doubling of faster growing bacteria.

In some embodiments, DL-Serine hydroxamate is added to the sample. In some embodiments, DL-Serine hydroxamate is added to the sample to prevent doubling of faster growing bacteria. In some embodiments, chloramphenicol is added to the sample to prevent doubling of faster growing bacteria.

### D-Amino Acid Labels

In some embodiments, the methods according to the present disclosure utilize labeled D-amino acids (DAAs), such as fluorescent D-amino acids (FDAAs). As used herein, “amino acid” or “amino acid probe” are used interchangeably to mean a molecule containing a first, or alpha, carbon attached to an amine group, a carboxylic acid group and a sidechain that is specific to each amino acid. A natural amino acid can include conventional elements such as carbon, hydrogen, oxygen, nitrogen and sulfur. An amino acid may be a naturally occurring amino acid or artificially created unnaturally occurring amino acid. Preferably, the amino acid is naturally occurring, and, unless otherwise limited, may encompass known analogues/synthetics of natural amino acids that can function in a similar manner as naturally occurring amino acids. With the exception of glycine, the natural amino acids all contain at least one chiral carbon atom. These amino acids therefore exist as pairs of stereoisomers (D- and L-isomers). Of particular interest herein are D-isomers or D-amino acids, particularly D-Ala, D-Asp, D-Cys, D-Glu and D-Lys, which are frequently found in the stem peptide of the PG unit.

It is well known in the art that amino acids within the same conservative group typically can substitute for one another without substantially affecting the function of a protein. For the purpose of the present disclosure, such conservative groups are based preferably on shared properties, as readily appreciated to those skilled in the art. See also, Alberts et al., “Small molecules, energy, and biosynthesis” 56-57 In: Molecular Biology of the Cell (Garland Publishing Inc. 3rd ed. 1994). Labeling of DAAs can be carried out by covalently attaching the label to a free amine group, such as free amine groups present on the sidechain that is specific to each amino acid. If the side chain lacks a free amine group, one of skill in the art understands how to add such groups, as is the case of adding such a group to D-Ala to obtain 3-amino-D-Ala. Some labels can be detected by using a labeled counter suitable for the detection of the label in question. In the examples below, 7-hydroxycoumarin 3-carboxylic acid (HCC—OH), 7-nitrobenzofurazan (NBD), 4-chloro-7-nitrobenzofurazan (NBD-C1), fluorescein (F) and carboxytetramethylrhodamine (T) were covalently attached to DAAs as labels.

Other coupling chemistries are known in the art that can be used for introducing labels into amino acids having functional groups other than an amine. Such amino acids include a functional alcohol group (e.g., serine and tyrosine), thiol group (e.g., cysteine), or carbonyl or carboxylate group (e.g., aspartate and glutamate). Such functional groups can be derivatized or reacted with suitably modified, activated coupling agents having labels of the types disclosed herein.

In some embodiments, the methods described herein use one or more of the following fluorescent amino acids: HADA, which is an HCC—OH-labeled 3-amino-D-Ala; NADA, which is an NBD-C1-labeled 3-amino-D-Ala; FDL, which is a F-labeled D-Lys; TDL, which is a T-labeled D-Lys; HDL, which is an HCC—OH-labeled D-Lys; NDL, which is an NBD-C1-labeled D-Lys; FADA, which is a F-labeled 3-amino-D-Ala; TADA, which is a T-labeled 3-amino-D-Ala. Other FDAAs can include a D-Glu having its side chain modified to include a free amine group linked to any of the fluorescent labels above (e.g., HADG, NADG, FADG and TADG). In some embodiments, any of the FDAAs referenced in U.S. Patent Application Publication No. 20/0024132, which is incorporated by reference in its entirety, are contemplated for use in any of the methods described herein.

In some embodiments, the mixture of live bacteria and the D-amino acid probe is incubated.

In some embodiments, the live bacteria sample and the D-amino acid is combined at about room temperature. In some embodiments, the mixture is incubated at between 15-40° C. In some embodiments, the mixture is incubated at between 20-40° C. In some embodiments, the mixture is incubated at between 25-40° C. In some embodiments, the mixture is incubated at between 30-40° C. In some embodiments, the mixture is incubated at between 15-25° C. In some embodiments, the mixture is incubated at between 18-20° C. In some embodiments, the mixture is incubated at 20° C. In some embodiments, the mixture is incubated at 25° C. In some embodiments, the mixture is incubated at 30° C. In some embodiments, the mixture is incubated at 37° C.

In some embodiments, the mixture is incubated for less than 45 minutes, between 1-45 minutes, less than 60 minutes, between 1-60 minutes, about 15 minutes, about 30 minutes, about 1 hour, about 90 minutes, less than about 90 minutes, about 2 hours, less than about 2 hours, about 3 hours, or less than about 3 hours.

In some embodiments, the live bacteria sample with the D-amino acid are added to a ferrofluid. In some embodiments, the samples are added to ferrofluid at a predetermined ratio, e.g., 1 part ferrofluid to 14 parts sample, 1 part ferrofluid to 10 parts sample, 1 part ferrofluid to 9 parts sample, 1 part ferrofluid to 8 parts sample, 1 part ferrofluid to 7 parts sample, 1 part ferrofluid to 6 parts sample, 1 part ferrofluid to 5 parts sample, 1 part ferrofluid to 4 parts sample, 1 part ferrofluid to 3 parts sample, 1 part ferrofluid to 2 parts sample, 1 part ferrofluid to 1 part sample, or 2 parts ferrofluid to 1 part sample.

### Click Chemistry

In some embodiments, the clickable bioorthogonal approach to the quantification of total live bacteria utilizing click chemistry involves the incorporation of a D-amino acid comprising a clickable bioorthogonal handle, wherein the clickable bioorthogonal handle is a moiety such as an alkyne, cyclooctyne, a nitrone or an azide group. In some embodiments, a fluorescent label is allowed to react with the bioorthogonal handle and the fluorescent tag comprises a corresponding reactive moiety, such as an azide, nitrone, alkyne, or cyclooctyne. Where the clickable bioorthogonal handle comprises an alkyne or cyclooctyne, the fluorescent label will comprise an azide or nitrone or similar reactive moiety. Where the bioorthogonal handle comprises an azide or nitrone, the fluorescent label will comprise a cyclooctyne, alkyne or similar reactive moiety. In some embodiments, any of the fluorophores described herein may be modified to contain a reactive group to serve as a fluorescent label in the methods described herein. The click chemistry reaction allows for the formation of a very stable covalent bond between the amino acid comprising the bioorthogonal handle and fluorophore label.

In some embodiments, the click reaction is a copper assisted click chemistry reaction which utilizes copper as a catalyst to perform the reaction.

In some embodiments, the click chemistry reaction is copper-free. The copper-free reaction uses ring strain in place of the copper catalyst to promote a [3+2] azide-alkyne cycloaddition reaction. For example, a cyclooctyne is a 8-carbon ring structure comprising an internal alkyne bond. The closed ring structure induces a substantial bond angle deformation of the acetylene, which is highly reactive with azide groups to form a triazole. Thus, cyclooctyne derivatives may be used for copper-free click reactions, without the toxic copper catalyst.

Another type of copper-free click reaction involves strain-promoted alkyne-nitrone cycloaddition. To address the slow rate of the original cyclooctyne reaction, electron-withdrawing groups are attached adjacent to the triple bond. Examples of such substituted cyclooctynes include difluorinated cyclooctynes, 4-dibenzocyclooctynol and azacyclooctyne. An alternative copper-free reaction involved strain-promoted alkyne-nitrone cycloaddition to give N-alkylated isoxazolines. The reaction was reported to have exceptionally fast reaction kinetics and was used in a one-pot three-step protocol for site-specific modification of peptides and proteins. Nitrones were prepared by the condensation of appropriate aldehydes with N-methylhydroxylamine and the cycloaddition reaction took place in a mixture of acetonitrile and water.

### Fluorophores

In some embodiments, the D-amino acid labels and fluorescent labels modified with a clickable bioorthogonal reactive group used according to methods of the present disclosure comprise one or more fluorescent dyes.

In some embodiments, D-amino acid labels and fluorescent labels modified with a clickable bioorthogonal reactive group used according to methods of the present disclosure comprise one or more fluorescent dyes including but not limited to cyanine, fluorescein, rhodamine, Alexa Fluor dyes, AZDye™ 633 Azide, DyLight fluors, ATTO Dyes, CF dyes such as picolyl azide dyes (Biotium), or any analogs or derivatives thereof.

In some embodiments, labels of the present invention include but are not limited to fluorescein and chemical derivatives of fluorescein; Eosin; Carboxyfluorescein; Fluorescein isothiocyanate (FITC); Fluorescein amidite (FAM); Erythrosine; Rose Bengal; Methylene blue; Laser dyes; Rhodamine dyes (e.g., Rhodamine, Rhodamine 6G, Rhodamine B, Rhodamine 123, Auramine O, Sulforhodamine 101, Sulforhodamine B, and Texas Red).

In some embodiments, labels of the present disclosure include but are not limited to ATTO dyes; Acridine dyes (e.g., Acridine orange, Acridine yellow); Alexa Fluor; 7-Amino actinomycin D; 8-Anilinonaphthalene-1-sulfonate; Auramine-rhodamine stain; Benzanthrone; 5,12-Bis(phenylethynyl) naphthacene; 9,10-Bis(phenylethynyl) anthracene; Blacklight paint; BODIPY; Brainbow; Calcein; Carboxyfluorescein; Carboxyfluorescein diacetate succinimidyl ester; Carboxyfluorescein succinimidyl ester; 1-Chloro-9,10-bis(phenylethynyl) anthracene; 2-Chloro-9,10-bis(phenylethynyl) anthracene; 2-Chloro-9,10-diphenylanthracene; Coumarin; Cyanine dyes (e.g., Cyanine such as Cy3 and Cy5, ZW800, ZW700, DiOC6, SYBR Green I); DAPI, Dark quencher, DyLight Fluor, Fluo-4, FluoProbes; Fluorone dyes (e.g., Calcein, Carboxyfluorescein, Carboxyfluorescein diacetate succinimidyl ester, Carboxyfluorescein succinimidyl ester, Eosin, Eosin B, Eosin Y, Erythrosine, Fluorescein, Fluorescein isothiocyanate, Fluorescein amidite, Indian yellow, Merbromin); Fluoro-Jade stain; Fura-2; Fura-2-acetoxymethyl ester; Green fluorescent protein, Hoechst stain, Indian yellow, Indo-1, Lucifer yellow, Luciferin, Merocyanine, Oxazin dyes (e.g., Cresyl violet, Nile blue, Nile red); Perylene; Phenanthridine dyes (Ethidium bromide and Propidium iodide); Phloxine, Phycobilin, Phycoerythrin, Phycoerythrobilin, Pyranine, Rhodamine, Rhodamine 123, Rhodamine 6G, RiboGreen, RoGFP, Rubrene, SYBR Green I, (E)-Stilbene, (Z)-Stilbene, Sulforhodamine 101, Sulforhodamine B, Synapto-pHluorin, Tetraphenyl butadiene, Tetrasodium tris(bathophenanthroline disulfonate) ruthenium (II), Texas Red, TSQ, Umbelliferone, or Yellow fluorescent protein.

In some embodiments, labels of the present invention include but are not limited to the Alexa Fluor family of fluorescent dyes (Molecular Probes, Oregon). Alexa Fluor dyes are typically used as cell and tissue labels in fluorescence microscopy and cell biology. The excitation and emission spectra of the Alexa Fluor series cover the visible spectrum and extends into the infrared. The individual members of the family are numbered according roughly to their excitation maxima (in nm). Alexa Fluor dyes are synthesized through sulfonation of coumarin, rhodamine, xanthene (such as fluorescein), and cyanine dyes. Sulfonation makes Alexa Fluor dyes negatively charged and hydrophilic. Alexa Fluor dyes are generally more stable, less prone to photobleaching, brighter, and less pH-sensitive than common dyes (e.g. fluorescein, rhodamine) of comparable excitation and emission, and to some extent the newer cyanine series. Exemplary Alexa Fluor dyes include but are not limited to Alexa Fluor-350, Alexa Fluor-405, Alexa Fluor-430, Alexa Fluor-488, Alexa Fluor-500, Alexa Fluor-514, Alexa Fluor-532, Alexa Fluor-546, Alexa Fluor-555, Alexa Fluor-568, Alexa Fluor-594, Alexa Fluor-610, Alexa Fluor-633, Alexa Fluor-635, Alexa Fluor-647, Alexa Fluor-660, Alexa Fluor-680, Alexa Fluor-700, or Alexa Fluor-750.

In some embodiments, labels of the present invention comprise one or more members of the DyLight Fluor family of fluorescent dyes (Dyomics and Thermo Fisher Scientific). Exemplary DyLight Fluor family dyes include but are not limited to DyLight-350, DyLight-405, DyLight-488, DyLight-549, DyLight-594, DyLight-633, DyLight-649, DyLight-680, DyLight-750, or DyLight-800.

In some embodiments, methods of the present invention comprise disrupting the outer membrane of gram-negative bacteria and exposing the peptidoglycan to ensure efficient delivery of the fluorescent dye (e.g. using agents that include, but are not limited to, organic solvents (ethanol, isopropanol, DMSO), 4% paraformaldehyde, or mild detergents (saponin, Triton-X, Tween, sodium dodecyl sulfate).

### Piper Platform

The present disclosure includes a means for automated quantification of the labeled bacteria cells in the sample, such as but not limited to, flow cytometry or ferrofluid-mediated cytometry using Ancera's PIPER platform, as manual microscopy methods for detecting fluorescently labeled bacteria are time consuming and labor intensive.

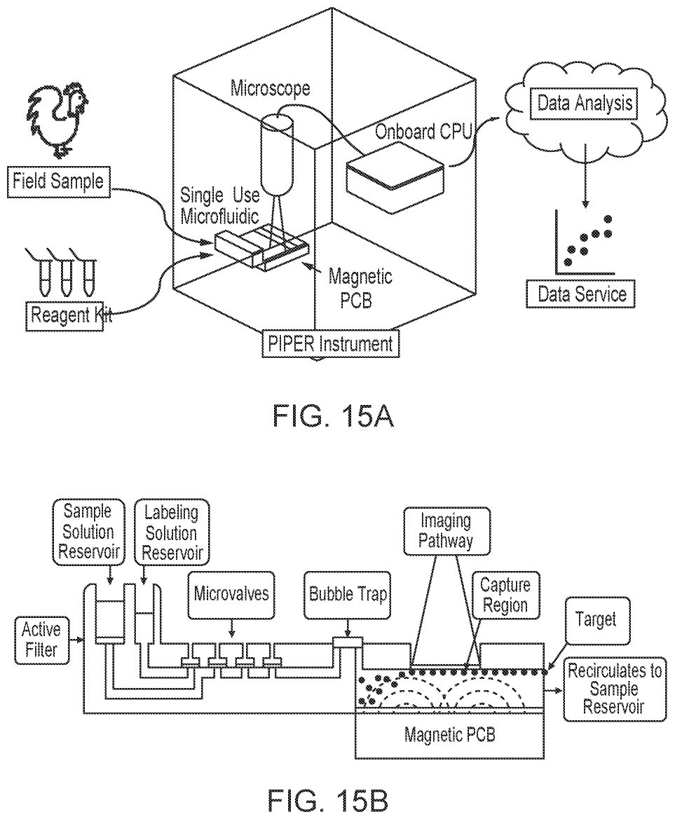

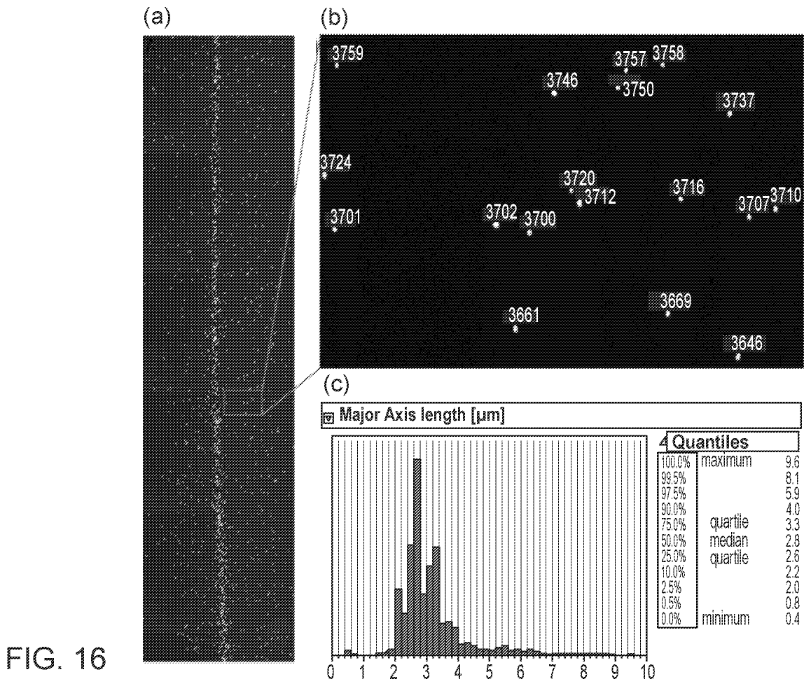

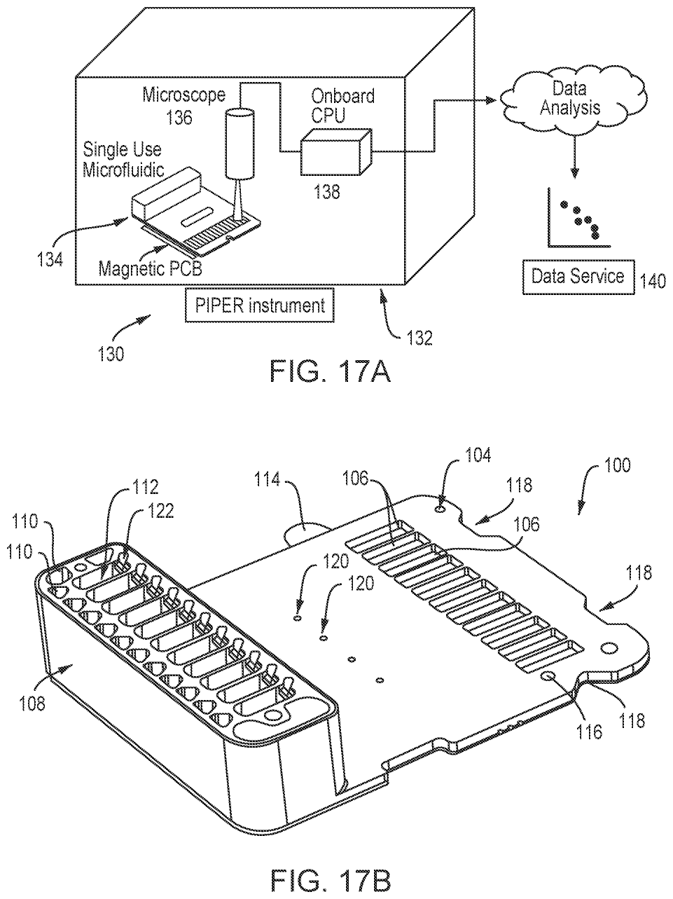

PIPER™ is an integrated platform composed of an instrument, disposable cartridge, and image analysis algorithm for automated cell counting. FIGS. 15A-B depict an overview of the PIPER platform. FIG. 15A shows a simplified PIPER workflow whereby a field sample is combined with a reagent kit before being loaded into one of twelve sample reservoirs on the microfluidic device. The microvalves in the cartridge pull sample over the magnetic PCB which pushes targets up for either or both of capture and imaging. Data can be transferred to a cloud-based system or a connected laboratory information management system. FIG. 15B shows the PIPER microfluidic flow path (portions of which are affected a cartridge device, see FIG. 17B) in which all components shown are included in the microfluidic consumable except the magnetic PCB. Sample solution is pumped with the microvalves through the active filter and into the detection channel above the magnetic PCB. In the detection channel, the magnetic field pushes targets into an imaging and/or capture region for antibody binding. During non-capture assays, the capture region is not coated but push up of particles suspended in the ferrofluid still occurs to concentrate and immobilize the targets for imaging.

The PIPER instrument uses a magnetic field generated by a printed circuit board (PCB) for non-contact manipulation of particles mixed with ferrofluid within a disposable cartridge. When the PCB is on, ferrofluid drives suspended particles, such as bacteria, to the top and center of the cartridge. This action leads to a dramatic increase in the concentration of cells in contact with the cartridge surface, ensuring high sensitivity across sample volumes (300 μl) that are larger than current samples used in conventional slide-based microscopy (typically <20 μl). When the flow is stopped, the targets are immobilized at the top of the cartridge purely by the action of ferrofluid and magnetic force (Kose and Koser, 2012), and a built-in fluorescent microscope with a digital camera above the cartridge obtains images of the immobilized samples. The ferrofluid provides a dark background, eliminating the need for extensive washes. An integrated bacterial detection algorithm automatically processes the scanned images, detects the labeled bacterial cells in the image, and quantifies the number of cells present. FIG. 16 depicts the image recognition algorithm on the PIPER system where (a) identified targets are marked by the algorithm, and (b) every target can be indexed, and its metadata are recorded for additional analysis (e.g., such as in (c) where the distribution of the major axis of the bacterial cells is plotted).

FIG. 17 illustrates a ferrofluidic assay system 130 including a ferrofluidic assay device 132 configured to receive a ferrofluidic cartridge 134 in a receiving area. The ferrofluidic cartridge 134 includes a plurality of windows each adjacent to an imaging zone in which one or more microbes can be visualized, an imager 136 (e.g., microscope(s)) configured to image each window of the cartridge either separately or together, a controller/processor/CPU 138 configured to control at least one of the ferrofluidic assay device, the ferrofluidic cartridge, and the imager, and assay processing components comprising at least one of reagents, and controls (not shown). The system is configured to at least one of: move or otherwise locate the bacteria to one or more of the windows. The data produced by the ferrofluidic assay system can be analyzed either on the assay device (e.g., using the processor), or via a data service 140.

Assay cartridges, for use in assay systems according to some embodiments, can include those as set out in published PCT application WO2018/026605 (incorporated herein by reference), as well as similar cartridges thereto. FIG. 17B illustrates a perspective view of a cartridge that is configured to perform multiple, independent, parallel assays (e.g., 2-10, 2-20, 2-100 or more, and ranges therebetween). Accordingly, the width of the cartridge 100 may change depending on the total number of assays supported.

As shown in FIG. 17B, assay cartridge 100 may comprise multiple layers integrated into a unitary/integral or an integrated cartridge (e.g., cartridge 100 may comprise a single construction with various features discussed below integrated therein). Cartridge 100 may include base layer 102, cartridge-instrument alignment features 118, a reagent spotting mask 114, pump valves 120 and a reservoir stack 108. Reservoir stack 108 may further include main reservoirs 112 (which can contain samples or mixtures of samples, regents, and the like), return chimneys 122 and a plurality of secondary (and, in some implementations, tertiary, etc.) reservoirs 110. The cartridge may also comprise internal alignment features 104 and 116 that may be used to ensure proper registration between the internal layers during its construction.

Cartridge-instrument alignment features 118 enable aligning placement of cartridge 100 within an assay instrument (not shown). The alignment may ensure, in part, that the cartridge main channels can align directly (or approximately) over the electrodes of an excitation PCB. This may also ensure that any other interface to the cartridge (such as pneumatic input ports for pumping fluid reagents within the cartridge) are aligned with the corresponding output from the instrument. Cartridge 100 may be inserted into an instrument slot (not shown) or may be placed at a designated space (such as a dedicated receptacle) within the assay instrument (not shown).

A plurality of cartridge analysis windows (or viewing ports) 106 are arranged to correspond with each of a plurality of reaction channels (not shown). The reaction channels within the cartridge may be embedded or formed over base 102. Cartridge analysis windows 106 provide optical viewing ports to each of the reaction channels.

The reagent spotting mask 114 may optionally be added to accommodate, for example, the precise positioning and spotting of assay reagents (e.g., capture reagents such as antibodies, aptamers, DNA fragments, other proteins or molecules used for surface modification or detection, etc.). The mask may consist of a matrix of patterned openings over an adhesive or a soft gasket (e.g., silicone rubber, PDMS, etc.) that is temporarily affixed over one of the bounding surfaces of the main assay channels. The assay reagents may thus be coated (or spotted) over that surface of the cartridge through the mask openings, either during the assembly of the cartridge or prior to running the assay by the end-user. Following an optional incubation period, the coated (or spotted) windows might be washed and/or dried, and the reagent spotting mask 114 may be removed (e.g., peeled off the cartridge surface) prior to capping the main assay channels with the final capping layer of the multi-stack assembly.

The internal alignment features 104 and 116 may optionally be used to assist in the assembly of the cartridge internal layers in order to ensure that each layer is properly aligned with and registered to its neighbors within a given positional tolerance. In some embodiments, the alignment features may be holes of a given shape (e.g., circular, square, hexagonal, diamond, etc.) that mate with alignment posts on an alignment jig.

The cartridge may have pneumatic input ports 120 which lead into pneumatic lines integrated into the cartridge. Together, they relay pressure and/or vacuum signals from the instrument to membrane valves (not shown) integrated into the body of the cartridge.

Reservoir stack 108 can retain the cartridge input fluids. For example, the reservoir stack 108 may receive and retain assay reagents which are then directed to the fluidic network (not shown) of cartridge 100. Main reservoirs 112 typically receive ferrofluid and/or input sample reagents that are intended for the ferrofluidic assay. They may also be configured to receive additional reagents, as needed.

Reservoir stack 108 may support more than one set of reservoir wells per independent assay. Secondary reservoirs 110 may be configured to receive secondary reagents used for an assay under study. The secondary reagents may include labels, dyes, secondary antibodies, PCR reagents required for DNA amplification after cell capture (for example), etc. In some implementations, the secondary reservoirs may be left blank or empty.

Additional structure for one and/or another of the embodiments can be found in one or more of the following disclosures: The current disclosure is related to U.S. Pat. Nos. 8,961,878, and 9,999,855, and PCT publication nos. WO2014144340, WO2014144782, WO2014144810, WO2014145765, WO2014165317, WO2016210348, WO2017004595, WO2018026605, WO2019117877, and WO2022/015845.

### LISTING OF NUMBERED EMBODIMENTS

Embodiment 1. A method for quantifying total live bacteria in a sample comprising:
obtaining a sample comprising a mixture of two or more types of live bacteria;
contacting the live bacteria with at least one D-amino acid probe under conditions sufficient for bacterial cell wall synthesis, wherein:
   the bacteria covalently incorporate the at least one D-amino acid probe,
   the amino acid probe includes a covalently attached fluorophore;
   adding the bacteria including the covalently incorporated D-amino acid probe to an assay processing device for at least counting cells;
and
detecting the labeled bacteria.

Embodiment 2. The method of embodiment 1, wherein the D-amino acid probe is a single amino acid or a dipeptide.

Embodiment 3. The method of embodiments 1 or 2, further comprising removing unincorporated D-amino acid probes from the sample.

Embodiment 4. The method of any of embodiments 1-3, wherein the labeled bacteria are detected via a fluorescence detector.

Embodiment 5. The method of any of embodiments 1-4, further comprising flowing the bacteria labeled with the D-amino acid probe toward an imaging region of the assay processing device, the imaging region including the fluorescence detector.

Embodiment 6. The method of any of embodiments 1-5, further quantifying a number of total live bacteria detected in the sample.

Embodiment 7. The method of any of embodiments 1-6, wherein the D-amino acid probe is selected from the group consisting of: HADA, BADA, NADA, FDL, TDL, HDL, NDL, FADA, TADA, HADG, NADG, FADG, and TADG.

Embodiment 8. A method for quantifying total live bacteria in a sample comprising:
obtaining a sample comprising live bacteria;
contacting the live bacteria with at least one D-amino acid probe under conditions sufficient for bacterial cell wall synthesis, wherein:
   the bacteria covalently incorporate the at least one D-amino acid probe, and
   the D-amino acid probe comprises a clickable bioorthogonal handle;
contacting the live bacteria with a fluorescent label comprising a bioorthogonal reactive group, wherein the clickable bioorthogonal reactive group forms a covalent bond with the clickable bioorthogonal handle; and
detecting the live bacteria.

Embodiment 9. The method of embodiment 8, wherein the D-amino acid probe comprises a single amino acid or a dipeptide.

Embodiment 10. The method of any one of embodiments 8 or 9, wherein the at least one D-amino acid probe comprises a combination of an amino acid and a dipeptide.

Embodiment 11. The method of any one of embodiments 8-10, wherein the clickable bioorthogonal reactive group comprises an azide, alkyne, or cycloalkyne group.

Embodiment 12. The method of any one of embodiments 8-11, wherein the clickable bioorthogonal reactive group comprises a cycloalkyne group.

Embodiment 13. The method of any one of embodiments 8-11, wherein the clickable bioorthogonal reactive group comprises an azide.

Embodiment 14. The method of any one of embodiments 8-11, wherein the clickable bioorthogonal reactive group comprises an alkyne.

Embodiment 15. The method of any one of embodiments 8-14, wherein the D-amino acid probe comprises an azide, alkyne, or cycloalkyne group.

Embodiment 16. The method of any of embodiments 8-14, wherein the D-amino acid probe comprises an azide group.

Embodiment 17. The method of any of embodiments 8-14, wherein the D-amino acid probe comprises an alkyne group.

Embodiment 18. The method of any of embodiments 8-17, wherein the D-amino acid probe comprises D-propargylglycine (EDA).

Embodiment 19. The method of any of embodiments 8-18, wherein the at least one D-amino acid probe comprises a combination of EDA and EDA-DA.

Embodiment 20. The method of any of embodiments 8-19, wherein the combination of EDA and EDA-DA is used at a ratio of from about 1:2, to about 2:1.

Embodiment 21. The method of any of embodiments 8-20, wherein the combination of EDA and EDA-DA is used at a ratio of about 1:1.

Embodiment 22. The method of any one of embodiments 8-21, further comprising removing the unincorporated D-amino acid probe from the sample.

Embodiment 23. The method of any one of embodiments 8-22, further comprising removing the unreacted fluorescent label from the sample.

Embodiment 24. The method of any of embodiments 8-23, wherein the labeled bacteria are detected via a fluorescence detector.

Embodiment 25. The method of any of embodiments 8-24, further comprising flowing the labeled bacteria including the at least one D-amino acid probe toward an imaging region of an assay device, the imaging region including the fluorescence detector.

Embodiment 26. The method of any of embodiments 8-25, further comprising quantifying the number of total live bacteria detected in the sample.

Embodiment 27. The method of any of embodiments 8-26, wherein the fluorescent label comprises an azide, alkyne, or cycloalkyne group.

Embodiment 28. The method of any of embodiments 8-27, wherein the fluorescent label includes an azide group.

Embodiment 29. The method of any of embodiments 8-28, wherein the fluorescent label comprises CF488 picolyl azide, AZDye 488 Picolyl Azide, CF633 picolyl azide, or AZDye™ 633 Azide.

Embodiment 30. The method of any one of embodiments 8-29, wherein the method further comprises a fixation step.

Embodiment 31. The method of any one of embodiments 8-30, wherein the fixation step comprises the addition of an organic solvent.

Embodiment 32. The method of any one of embodiments 8-31, wherein the organic solvent is ethanol.

Embodiment 33. The method of any one of embodiments 8-32, wherein the fixation step is followed by an incubation period.

Embodiment 34. The method of any one of embodiments 8-33, wherein the incubation period is from about 1 minute, to about 30 minutes.

Embodiment 35. The method of any one of embodiments 8-34, wherein the incubation period is about 1 minute, about 2 minutes, about 3 minutes, about 4 minutes, about 5 minutes, about 7 minutes, about 10 minutes, or about 15 minutes.

Embodiment 36. The method of any one of embodiments 8-35, wherein the clickable bioorthogonal handle of the D-amino acid probe reacts with the clickable bioorthogonal reactive group of the fluorescent label to form a 1,2,3-triazole.

Embodiment 37. The method of any one of embodiments 8-36, wherein the reaction between the D-amino acid and the fluorescent label is a copper catalyzed click chemistry reaction.

Embodiment 38. The method of any one of embodiments 8-37, wherein the reaction between the D-amino acid and the fluorescent label is a copper-free reaction.

Embodiment 39. The method of any one of embodiments 8-38, wherein the reaction between the D-amino acid and the fluorescent label is a copper-free strain promoted click chemistry reaction.

Embodiment 40. The method of any one of embodiments 1-39, wherein the contacting the live bacteria with at least one D-amino acid probe comprises incubating the live bacteria with the at least one D-amino acid probe.

Embodiment 41. The method of any one of embodiments 1-40, wherein the contacting the live bacteria with at least one D-amino acid probe occurs for between about 10 and about 120 minutes.

Embodiment 42. The method of any one of embodiments 1-41, wherein the contacting the live bacteria with at least one D-amino acid probe occurs for at least about 10 minutes, at least about 20 minutes, at least about 30 minutes, at least about 40 minutes, at least about 50 minutes, at least about 60 minutes, at least about 70 minutes, at least about 80 minutes, at least about 90 minutes, at least about 100 minutes, at least about 110 minutes, or at least about 120 minutes.

Embodiment 43. The method of any one of embodiments 1-42, wherein the contacting the live bacteria with at least one D-amino acid probe occurs for between about 20 and about 90 minutes.

Embodiment 44. The method of any one of embodiments 1-43, wherein the contacting the live bacteria with at least one D-amino acid probe comprises incubating the live bacteria with the at least one D-amino acid probe for between about 20 and about 90 minutes.

Embodiment 45. The method of any one of embodiments 1-44, wherein the sample is supplemented with a cell metabolism booster.

Embodiment 46. The method of embodiment 45, wherein the cell metabolism booster comprises at least one of glucose and sodium pyruvate.

Embodiment 47. The method of any one of embodiments 1-46, wherein the sample is supplemented with a small molecule that controls cell doubling.

Embodiment 48. The method of embodiment 47, wherein the small molecule comprises DL-serine hydroxamate or chloramphenicol.

Embodiment 49. The method of any one of embodiments 1-48, wherein the sample is supplemented with at least one of glucose and sodium pyruvate.

Embodiment 50. The method of any one of embodiments 1-49, wherein quantifying the total live bacteria in the sample is performed using a microfluidic device.

Embodiment 51. The method of any one of embodiments 1-50, wherein quantifying the total live bacteria in the sample is performed using flow cytometry

Embodiment 52. The method of any one of embodiments 1-51, wherein quantifying the total live bacteria in the sample is performed using a ferrofluid-based microfluidic device.

Embodiment 53. The method of any one of embodiments 1-52, wherein the sample is obtained from a food processing plant.

Embodiment 54. The method of any one of embodiments 1-53, wherein the sample is obtained from a sample from a beef or poultry processing plant.

Embodiment 55. The method of any one of embodiments 1-54, wherein the sample is obtained from a poultry processing plant.

Embodiment 56. The method of any one of the previous embodiments, wherein the sample is obtained from a carcass.

Embodiment 57. The method of any one of embodiments 1-56, wherein the method does not include culturing the bacteria before analysis.

Embodiment 58. The method of any one of embodiments 1-57, wherein the quantifying step comprises counting individually labeled bacteria cells.

Embodiment 59. The method of any one of embodiments 1-58, wherein the live bacteria sample comprises gram negative bacteria.

Embodiment 60. The method of any one of embodiments 1-59, wherein the live bacteria is selected from the group consisting of: Aeromonas hydrophila, Bukholderia cenocepacia, Campylobacter jejuni, Citrobacter freundii, Enterobacter sakasakii (Cronobacter), Escherichia coli, Flavobacterium Sp., Hafnia alvei, Klebsiella pneumoniae, Kluyvera Sp., Moraxella catarrhalis, Proteus mirabilis, Pseudomonas aeruginosa, Salmonella Typhimurium, Serratia liquefaciens, Shewanella putrefaciens, Shigella flexneri, Vibrio parahaemolyticus, and Yersinia enterocolitica.

Embodiment 61. The method of any one of embodiments 1-60, wherein the live bacteria sample comprises gram positive bacteria.

Embodiment 62. The method of any one of embodiments 1-61, wherein the bacteria is selected from the group consisting of: Bacillus cereus, Bacteroides fragilis, Brochothrix thermosphacta, Clostridium perfringens, Corynebacterium sp, Enterococcus faecalis, Lactobacillus brevis, Lactococcus lactis, Leuconostoc lactis, Listeria monocytogenes, Staphylococcus aureus, Streptococcus pyogenes, and Weissella viridescens.

Embodiment 63. The method of any one of embodiments 1-62, wherein the bacteria is selected from the group consisting of: Acinetobacter geminorum, Acinetobacter haemolyticus, Acinetobacter lwoffii, Acinetobacter pittii or A. calcoaceticus, Bacillus licheniformis, Bacillus mojavensis, Brachybacterium paraconglomeratum, Chryseobacterium gambrini, Corynebacterium ammoniagenes, Coryneobacterium callunae, Cytobacillus solani, Empedobacter falsenii, Exiguobacterium indicum, Heyndrickxia oleronia, Kocuria tytonicola; or K. tytonis, Kurthia gibsonii, Kurthia populi, Macrococcus caseolyticus, Mammaliicoccus lentus, Microbacterium invictum, Microbacterium maritypicum, Microbacterium testaceum, Pseudomonas mosselii, Pseudoxanthomonas mexicana, Rothia nasimurium, Staphylococcus pseudoxylosus, Staphylococcus lloydii; or S. kloosii, Staphylococcus nepalensis, Staphylococcus ureilyticus, and Streptococcus pluranimalium.

Embodiment 64. The method of any one of embodiments 1-63, wherein the sample comprises both gram negative and gram positive bacteria.

Embodiment 65. The method of any one of embodiments 8-64, wherein the method further comprises a fixation step prior to the contacting of live bacteria with a fluorescent label.

Embodiment 66. A quantification system configured to quantify fluorescently labeled live bacteria from a sample according to the method of any one of embodiments 1-64, comprising:
a ferrofluidic assay device configured to receive a microfluidic cartridge containing a sample;
the microfluidic cartridge includes a plurality of microfluidic channels;
each microfluidic channel contains an imaging window;
an imager configured to image each window of the cartridge either separately or together;
a controller configured to control at least one of the ferrofluidic assay device, the microfluidic cartridge containing sample mixed with ferrofluid, and the imager; and
assay processing components comprising at least one of reagents, and controls;
wherein the system is configured to at least one of moving or otherwise locating the labeled bacteria to one or more of the windows where they can be any and all of imaged and quantified.

### Example 1: Demonstration of the Versatility and Efficacy of 3-Step Metabolic Labeling

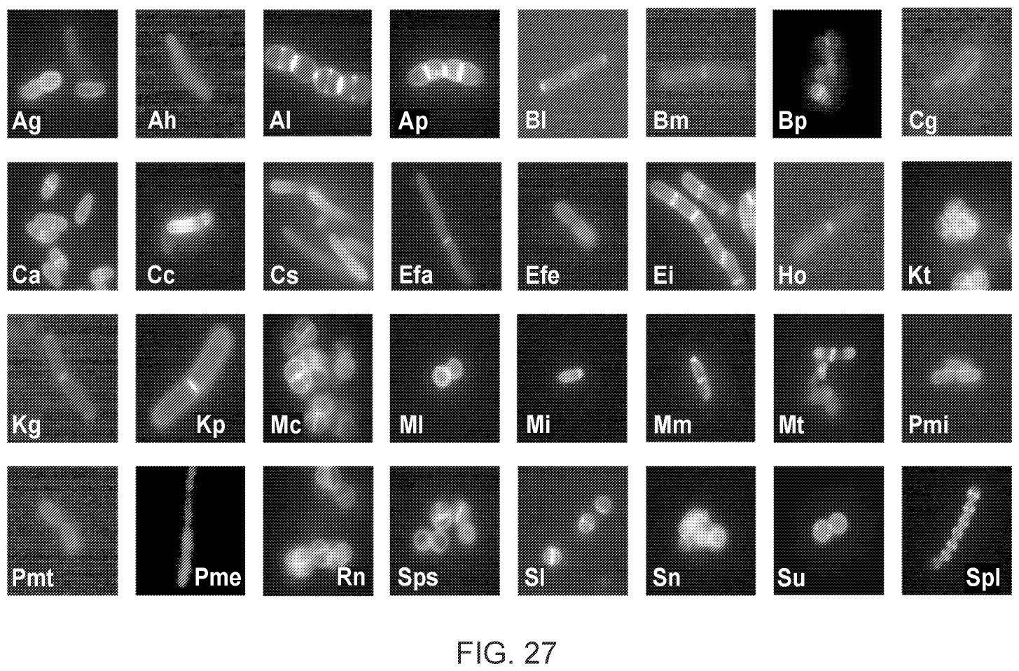

Versatility and efficacy of 3-step metabolic labelling was demonstrated on 32 (thirty-two) species isolated from a poultry processing plant. The species identities, phylogenetic standing, and sources are indicated in Table 1. Abbreviations are for FIG. 27. Comparison of images collected by conventional microscopy revealed the variety of cell morphologies, clustering and labeling patterns that can be detected by the 3-step metabolic labelling (FIG. 27).

Experimental detail: Swab samples were collected using sponge sticks (3M, Saint Paul, MN) soaked in Letheen broth or Neutralizing buffer. Various areas at the pre-chill poultry processing facility, including evisceration equipment, floor drains and ice maker, were swabbed as per manufacturer instructions, and placed on ice. All samples were processed within 24 hours after collection. Eluents from the sponges were aseptically diluted in sterile 1× PBS (phosphate buffered saline) and 0.1 mL of each dilution was spread on TSA (tryptic soy agar) plate. Plates were incubated for 48 hours at 37° C. to allow for colonies to develop. Individual colonies with various morphologies were picked and isolated on fresh TSA plates using standard microbiological techniques. Each strain was subcultured in BHI broth and banked at −80° C. for long-term storage.

Commercial 16S rDNA sequencing analysis was used to establish the identity of the isolates in Table 1, listed in alphabetical order. The sequencing revealed 32 distinct species (some isolated more than once) from 23 different genera. Those represented four major phylogenetic groups, in order of abundance: Firmicutes, Gammaproteobacteria, Actinobacteria, and Bacteroidetes.

All isolates were tested by the 3-step labeling protocol and visualized using conventional microscopy, with representative images shown in FIG. 27. In short, an initial culture of each strain was obtained by inoculating a single colony into BHI and placing it into a 37° C. incubator overnight. On the next day, the culture was diluted 100-fold into fresh BHI and allowed to recover at 37° C. for 1 hour prior to addition of 1 mM EDA. After a 90 minute incubation at 37° C., cells were harvested by centrifugation (10 min 10,000 g), and supernatant containing media with excess (unincorporated) EDA was discarded. Cell pellets were resuspended in 0.75 mL of 1×PBS and mixed with 0.25 mL of 96% Ethanol. After a 5 minute incubation at room temperature, cells were pelleted again by centrifugation (10 min 10,000 g) and supernatant containing media with ethanol and residual EDA was discarded.

The CuAAC click reaction was carried out by resuspending cell pellets in 0.25 mL of 1×PBS solution containing 200 μM copper sulfate (II) (MilliporeSigma, Burlington, MA), 128 μM THPTA ligand (Lumiprobe, Hunt Valley, MD), 2.5 mM Ascorbic acid (MilliporeSigma, Burlington, MA), 25 μM CF488 picolyl azide fluorescent dye (Biotium, Fremont, CA), and 5% DMSO (Thermo Fisher Scientific, Waltham, MA). After a 30-minute incubation at room temperature, 1 mL of 1×PBS was added to each reaction, and cells were harvested by centrifugation (10 min 10,000 g). Supernatant containing excess dye and reaction components was discarded, cells were washed with 1 mL of 1×PBS, and cells were pelleted again by centrifugation. Finally, each cell pellet was resuspended in 0.1 mL of 1×PBS and visualized under an A1 Fluorescence Microscope (Zeiss, Germany) using an Apochromat 63× oil objective. Images were captured with an Olympus EP50 camera installed in a Zeiss AXIO Imager. Brightness and contrast of each image were adjusted to highlight cell morphologies and labeling patterns.

| Abbreviation | | Phylogenetic standing | |
|---|---|---|---|
| for FIG._27 | ID | (phylum; class; order; family) | source |
| Ag | Acinetobacter | Proteobacteria; | Manual eviscerator |
| | geminorum | Gammaproteobacteria; | drain |
| | | Pseudomonadales; | |
| | | Moraxellaceae | |
| Ah | Acinetobacter | Proteobacteria; | Floor drain cover |
| | haemolyticus | Gammaproteobacteria; | |
| | | Pseudomonadales; | |
| | | Moraxellaceae | |
| Al | Acinetobacter | Proteobacteria; | Floor drain cover |
| | lwoffii | Gammaproteobacteria; | |
| | | Pseudomonadales; | |
| | | Moraxellaceae | |
| Ap | Acinetobacter pittii | Proteobacteria; | Manual eviscerator |
| | or A. calcoaceticus | Gammaproteobacteria; | drain |
| | | Pseudomonadales; | |
| | | Moraxellaceae | |
| Bl | Bacillus | Firmicutes; Bacilli; | Manual eviscerator |
| | licheniformis | Bacillales; Bacillaceae | trough |
| Bm | Bacillus mojavensis | Firmicutes; Bacilli; | Ice maker floor |
| | | Bacillales; Bacillaceae | |
| Bp | Brachybacterium | Actinobacteria; | Automated eviscerator |
| | paraconglomeratum | Actinobacteria; | pan |
| | | Micrococcales; | |
| | | Dermabacteraceae | |
| Cg | Chryseobacterium | Bacteroidotes; Flavobacteriia; | Floor drain cover |
| | gambrini | Flavobacteriales; | |
| | | Weeksellaceae | |
| Ca | Corynebacterium | Actinobacteria; | Automated eviscerator |
| | ammoniagenes | Actinobacteria; | pan |
| | | Mycobacteriales; | |
| | | Corynebacteriaceae | |
| Ca | Coryneobacterium | Actinobacteria; | Floor drain cover |
| | callunae | Actinobacteria; | |
| | | Mycobacteriales; | |
| | | Corynebacteriaceae | |
| Cs | Cytobacillus solani | Firmicutes; Bacilli; | Floor drain cover |
| | | Bacillales; Bacillaceae | |
| Efa | Empedobacter | Bacteroidotes; Flavobacteriia; | Floor drain cover |
| | falsenii | Flavobacteriales; | |
| | | Weeksellaceae | |
| Efe | Escherichia | Proteobacteria; | Floor drain cover |
| | fergusonii or | Gammaproteobacteria; | |
| | Shigella flexneri | Enterobacterales; | |
| | | Enterobacteriaceae | |
| Ei | Exiguobacterium | Firmicutes; Bacilli; | Manual eviscerator |
| | indicum | Bacillales; Bacillaceae | trough |
| Ho | Heyndrickxia | Firmicutes; Bacilli; | Manual eviscerator |
| | oleronia | Bacillales; Bacillaceae | trough |
| Kt | Kocuria tytonicola; | Actinobacteria; | Automated eviscerator |
| | or K. tytonis | Actinobacteria; | spindles |
| | | Micrococcales; | |
| | | Micrococcaceae | |
| Kg | Kurthia gibsonii | Firmicutes; Bacilli; | Floor drain cover |
| | | Bacillales; Planococcaceae | |
| Kp | Kurthia populi | Firmicutes; Bacilli; | Floor drain cover |
| | | Bacillales; Planococcaceae | |
| Mc | Macrococcus | Firmicutes; Bacilli; | Manual eviscerator |
| | caseolyticus | Bacillales; Staphylococcaceae | drain and floor |
| | | | drain cover |
| Ml | Mammaliicoccus | Firmicutes; Bacilli; | Automated eviscerator |
| | lentus | Bacillales; Staphylococcaceae | spindles |
| Mi | Microbacterium | Actinobacteria; | Ice maker chute |
| | invictum | Actinobacteria; | |
| | | Micrococcales; | |
| | | Microbacteriaceae | |
| Mm | Microbacterium | Actinobacteria; | Ice maker chute |
| | maritypicum | Actinobacteria; | |
| | | Micrococcales; | |
| | | Microbacteriaceae | |
| Mt | Microbacterium | Actinobacteria; | Ice maker chute |
| | testaceum | Actinobacteria; | |
| | | Micrococcales; | |
| | | Microbacteriaceae | |
| Pmi | Proteus mirabilis | Proteobacteria; | Manual eviscerator |
| | | Gammaproteobacteria; | trough |
| | | Enterobacterales; | |
| | | Enterobacteriaceae | |
| Pmo | Pseudomonas | Proteobacteria; | Manual eviscerator |
| | mosselii | Gammaproteobacteria; | drain |
| | | Pseudomonadales; | |
| | | Pseudomonadaceae | |
| Pme | Pseudoxanthomonas | Proteobacteria; | Ice maker chute |
| | mexicana | Gammaproteobacteria; | |
| | | Xanthomonadales; | |
| | | Xanthomonadaceae | |
| Rn | Rothia nasimurium | Actinobacteria; | Automated eviscerator |
| | | Actinobacteria; | pan |
| | | Micrococcales; | |
| | | Micrococcaceae | |
| Sps | Staphylococcus | Firmicutes; Bacilli; | Automated eviscerator |
| | pseudoxylosus | Bacillales; Staphylococcaceae | pan and manual |
| | | | eviscerator trough |
| Sl | Staphylococcus | Firmicutes; Bacilli; | Automated eviscerator |
| | lloydii; or S. kloosii | Bacillales; Staphylococcaceae | pan |
| Sn | Staphylococcus | Firmicutes; Bacilli; | Manual eviscerator |
| | nepalensis | Bacillales; Staphylococcaceae | trough |
| Su | Staphylococcus | Firmicutes; Bacilli; | Automated eviscerator |
| | ureilyticus | Bacillales; Staphylococcaceae | spindles |
| Spl | Streptococcus | Firmicutes; Bacilli; | Automated eviscerator |
| | pluranimalium | Lactobacillus; | pan |
| | | Streptococcaceae | |

### Example 2: Three-Step Labeling Using EDA in Combination with Dipeptide EDA-DA

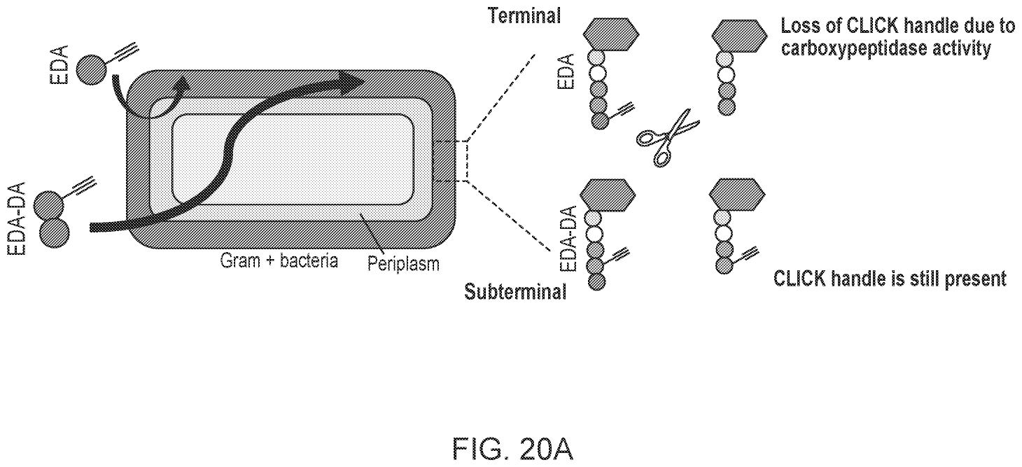

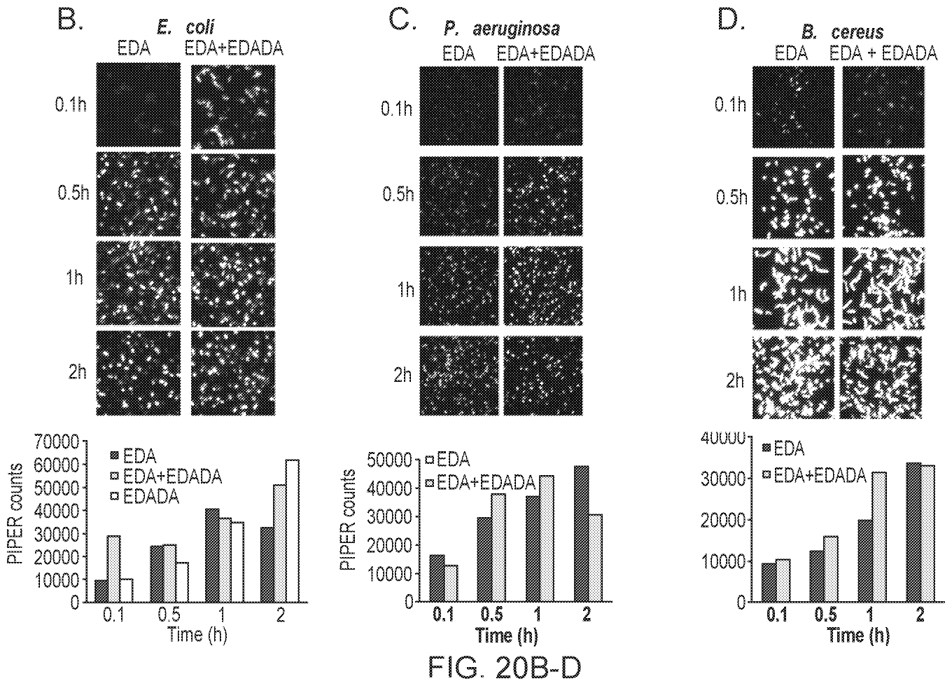

While the use of EDA-DA alone had a negative impact on cell labeling, use of a 1:1 mixture of EDA and EDA-DA benefited some strains. FIG. 20A depicts the use of EDA-DA. EDA is incorporated in the periplasm by enzymes outside the cytoplasmic cell membrane at the TERMINAL position of the peptidoglycan pentapeptide, while EDA-DA is incorporated by the intracellular enzymes during synthesis of the peptidoglycan (PG) precursor at a SUBTERMINAL position in the pentapeptide. Cellular transpeptidase/carboxypeptidases can cleave the terminal D-amino acid, leading to the loss of signal, but not the subterminal D-amino acids.

Experimental Detail: Prior to 3-step labeling, a single colony of the selected bacterial strain was inoculated into BHI and incubated overnight at 37° C. To carry out the 1^{st} step, approximately 3e7 CFU of bacteria from the overnight culture were added to 3 mL of fresh BHI supplemented with 1 mM EDA, 1 mM EDA-DA, or a mixture of 0.5 mM EDA and 0.5 mM EDA-DA and incubated for 0.1 h, 0.5 h, 1 h, and 2 h at 37° C. For the 2^{nd} step, cells were normalized to be at 3e7 CFU in 2.5 mL, and for each treatment group 225 μL of the cells were mixed with 75 μL of 96% ethanol in 1.7 mL tubes. After a 5-minute incubation at room temperature, cells were pelleted by centrifugation (10 min at 10,000 g) and supernatant containing media with ethanol and residual D-amino acids was discarded. Cells were then resuspended in 300 μL of 1×PBS and pelleted again by centrifugation (10 min at 10,000 g). The 3^{rd} step was carried out by resuspending cell pellets in 0.25 mL of 1×PBS solution containing 200 μM copper sulfate (II) (MilliporeSigma, Burlington, MA), 400 μM THPTA ligand (Lumiprobe, Hunt Valley, MD), 2.5 mM Ascorbic acid (MilliporeSigma, Burlington, MA), 10 μM CF488 picolyl azide fluorescent dye (Biotium, Fremont, CA), and 5% DMSO (Thermo Fisher Scientific, Waltham, MA). After a 30-minute incubation at room temperature, 500 μL of 1×PBS was added to each reaction, and cells were harvested by centrifugation (10 min at 10,000 g). Supernatant containing excess dye and reaction components was discarded, cells were washed with 500 μL of 1×PBS, and the cells were pelleted again by centrifugation. Finally, each cell pellet was resuspended in 0.3 mL of 1×PBS and mixed with an equal volume of ferrofluid. All samples were run on PIPER and cell counts were generated by image analysis using an automated bacterial image recognition algorithm.

FIG. 20B depicts a PIPER image of E. coli (ATCC 35421) cells (top) and PIPER counts (bottom) after 0.1, 0.5, 1, or 2 hours of metabolic labeling with EDA alone, a 1:1 mixture of EDA and EDA-DA, or EDA-DA alone. An automated bacterial image recognition algorithm determined labeled cell counts. FIG. 20C depicts a PIPER image of P. aeruginosa (ATCC 27853) cells (top) and PIPER counts (bottom) after 0.1, 0.5, 1, or 2 hours of metabolic labeling with EDA alone, or a 1:1 mixture of EDA and EDA-DA. An automated bacterial image recognition algorithm determined labeled cell counts. FIG. 20D depicts a PIPER image of B. cereus (ATCC 33019) cells (top) and PIPER counts (bottom) after 0.1, 0.5, 1, or 2 hours of metabolic labeling with EDA alone or a 1:1 mixture of EDA and EDA-DA. An automated bacterial image recognition algorithm determined labeled cell counts.

### Example 3: Introduction of an Ethanol Treatment Step Prior to Click Labeling

Experimental Detail: A single colony of each indicated bacteria was inoculated into BHI and incubated overnight at 37° C. On the next day, the cells were diluted into fresh BHI and incubated for 2 h at 37° C. followed by an additional 90-minute incubation in the presence or absence of 1 mM EDA, where indicated. Where indicated, an aliquot of each culture was mixed with 96% ethanol to a final concentration of 70% (A, B) or 25% (C) and incubated for 5 to 10 min at room temperature. Cultures not exposed to ethanol were incubated for an equal amount of time in 1×PBS. Cells were pelleted by centrifugation for 10 min at 10,000 g, and supernatant containing excess EDA and ethanol was discarded. Cells were then resuspended in 300 μL of 1×PBS and pelleted again by centrifugation (10 min at 10,000 g). The 3^{rd} step was carried out by resuspending cell pellets in 0.25 mL of 1×PBS solution containing 200 μM copper sulfate (II) (MilliporeSigma, Burlington, MA), 128 μM THPTA ligand (Lumiprobe, Hunt Valley, MD), 2.5 mM Ascorbic acid (MilliporeSigma, Burlington, MA), 25 μM CF488 picolyl azide fluorescent dye (Biotium, Fremont, CA), and 5% DMSO (Thermo Fisher Scientific, Waltham, MA). After a 30-minute incubation at room temperature, 500 μL of 1×PBS was added to each reaction, and cells were harvested by centrifugation (10 min at 10,000 g). Supernatant containing excess dye and reaction components was discarded, cells were washed with 500 μL of 1×PBS, and the cells were pelleted again by centrifugation. Finally, each cell pellet was resuspended in 0.3 mL of 1×PBS and mixed with an equal volume of ferrofluid. All samples were run on PIPER, and cell counts were generated by image analysis using an automated bacterial image recognition algorithm.

FIG. 21 shows that introduction of an ethanol treatment step prior to click labeling improves detection of some Gram-negative species. A short incubation (5 or 10 minutes) with ethanol prior to the click reaction dramatically improved detection of E. coli (ATCC 35421), but only if the cells were grown in the presence of EDA. In the control samples (no EDA), cells were incubated with 1×PBS for an equal amount time (FIG. 21A). CuAAC labeling of E. coli, a gram-negative bacteria, was poor in the absence of ethanol treatment, while the labeling of B. cereus (ATCC 33019), a gram-positive bacteria, occurred efficiently both with or without ethanol treatment (FIG. 21B). Ethanol treatment prior to CuAAC labeling improved cell counts and/or cell mean intensity and reduced the variability across technical replicates (N=3) for the four Gram-negative bacteria tested: Salmonella enterica (ATCC 12023), Klebsiella pneumoniae (ATCC 13882), Citrobacter freundii (ATCC 8090), and E. coli (ATCC 35421) (FIG. 21C).

### Example 4: Detection of Live Bacteria Spiked into Poultry Carcasses Using the Strain Promoted Alkyne-Azide Cycloaddition (SPAAC) Variant of the 3-Step Metabolic Labeling

Experimental Detail: Whole carcass rinsate samples were prepared as per FSIS USDA guidance using two separate whole chickens from the grocery store obtained prior to the “use by” date. Sodium polyanethol sulfonate was added to 0.05% to limit blood clotting. Prior to 3-step metabolic labeling, an aliquot from each rinsate was removed for serial dilution and plating on APC petrifilm (3M, Saint Paul, MN) and incubated as per manufacturer instructions to evaluate the number of total viable bacteria by the “gold standard” method. 1 mL rinsate aliquots were used for incubation with or without an enumerated culture of Salmonella enterica (SE; NCTC 12033). As a control, the same number of SE cells was spiked into BPW media. For SPAAC samples, the 1^{st} step of labeling was carried out by adding 5 μg/mL chloramphenicol and 1 mM ADA (3-Azido-D-alanine HCl, Jena Bioscience, Germany). For CuAAC samples, the 1^{st} step of labeling was carried out by adding 5 μg/mL chloramphenicol and 1 mM EDA. Next, samples were incubated at 37° C. for 90 minutes. 5 mg/ml of α-chymotrypsin from bovine pancreas (MilliporeSigma, Burlington, MA) was added during the last 30 minutes of incubation to break up protein aggregates. Cells were harvested by centrifugation (10 min 10,000 g), and supernatant was discarded. The 2^{nd} step was carried out by resuspending the pellets in 0.75 mL of 1×PBS and mixing them with 0.25 mL of 96% ethanol. After a 5-minute incubation at room temperature, cells were pelleted by centrifugation (10 min 10,000 g) and supernatant containing media with ethanol and residual DAAs was discarded. For SPAAC, the 3^{rd} step was carried out by resuspending cell pellets in 0.25 mL of 1×PBS solution containing 10 μM DBCO-AF488 (Jena Bioscience, Germany), and 5% DMSO (Thermo Fisher Scientific, Waltham, MA). For CuAAC, the 3^{rd} step was carried out by resuspending cell pellets in 0.25 mL of 1×PBS solution containing 0.8 mM copper sulfate (II) (MilliporeSigma, Burlington, MA), 1.6 mM THPTA ligand (Lumiprobe, Hunt Valley, MD), 10 mM Ascorbic acid (MilliporeSigma, Burlington, MA), 5% DMSO (Thermo Fisher Scientific, Waltham, MA), and 10 μM CF488 picolyl azide (Biotium, Fremont, CA). After a 30-minute incubation at room temperature, 1 mL of 1×PBS was added to each reaction, and cells were harvested by centrifugation (10 min 10,000 g). Supernatant containing excess dye and reaction components was discarded, and the cells were washed with 1 mL of 1×PBS and pelleted again by centrifugation. Finally, each cell pellet was resuspended in 0.15 mL of 1×PBS and mixed with an equal volume of ferrofluid. All samples were run on PIPER, and cell counts were generated by image analysis using an automated bacterial image recognition algorithm.

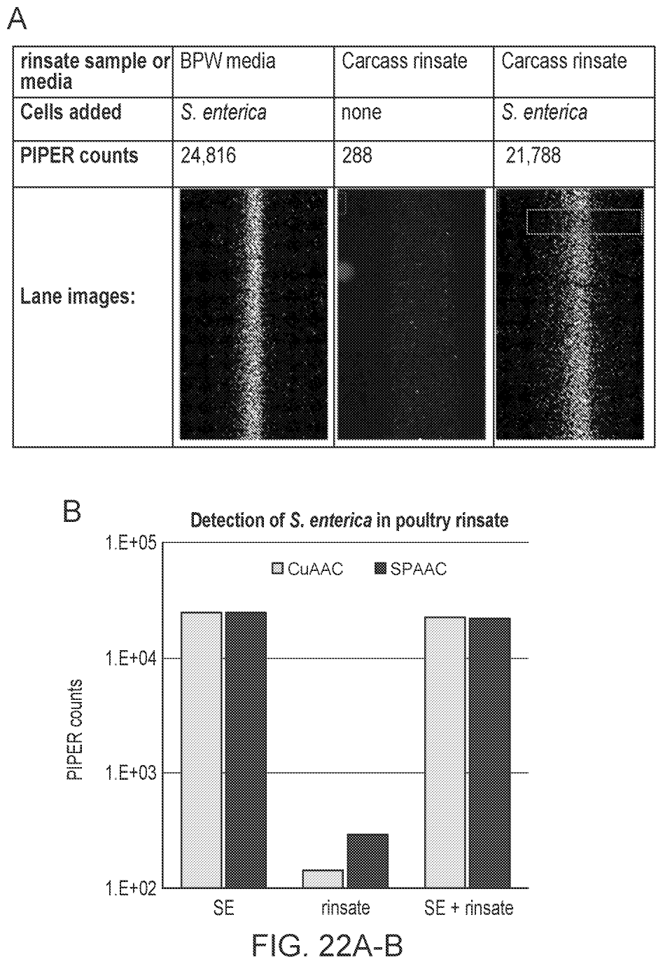

FIG. 22 depicts the strain promoted alkyne-azide cycloaddition (SPAAC) variant of the 3-step metabolic labeling detects live bacteria spiked into poultry carcasses. FIG. 22A depicts PIPER images of a carcass rinsate sample or media-only control with or without added Salmonella enterica (SE) cells subjected to 3-step metabolic labeling using the strain promoted alkyne-azide cycloaddition (SPAAC) reaction. FIG. 22B depicts the comparison of Salmonella enterica (SE) cell detection in carcass rinsates by 3-step metabolic labeling using CuAAC (grey bars) or SPAAC (black bars). Cell detection in the whole carcass rinsate samples (“SE+rinsate”) is as efficient as when cells are tested in media only (“SE”) by either method. PIPER counts in the unspiked rinsate control samples (“rinsate”) are within system noise due to imperfections of the image algorithm that is still in development. APC of the rinsate confirmed that the innate bacterial population of the tested rinsate sample was below 100 CFU/mL.

### Example 5: Detection of Live Bacteria Spiked into Poultry Carcasses Using the 3-Step Metabolic Labeling

Experimental Detail: Whole carcass rinsate samples were prepared as per FSIS USDA guidance using two separate whole chickens from the grocery store obtained prior to the “use by” date. Sodium polyanethol sulfonate was added to 0.05% to limit blood clotting. Prior to 3-step metabolic labeling, an aliquot from each rinsate was removed for serial dilution and plating on APC petrifilm (3M, Saint Paul, MN) and incubated as per manufacturer instructions to evaluate the number of total viable bacteria by the “gold standard” method. 5 mL rinsate aliquots were used for incubation with or without an enumerated culture of Klebsiella pneumoniae (ATCC 13882). As a control, the same number of KP cells was spiked into BPW media. The 1st step of labeling was carried out by adding 5 μg/mL chloramphenicol and 1 mM EDA, where indicated, followed by a 90-minute incubation at 37° C. 5 mg/ml of α-chymotrypsin from bovine pancreas (MilliporeSigma, Burlington, MA) was added during the last 30 minutes of incubation to break up protein aggregates. Cells were harvested by centrifugation (10 min 10,000 g), and supernatant was discarded. The 2nd step was carried out by resuspending the pellets in 0.75 mL of 1×PBS and mixing them with 0.25 mL of 96% ethanol. After a 5-minute incubation at room temperature, cells were pelleted by centrifugation (10 min 10,000 g) and supernatant containing media with ethanol and residual EDA was discarded. The 3rd step was carried out by resuspending cell pellets in 0.25 mL of 1×PBS solution containing 2 mM copper sulfate (II) (MilliporeSigma, Burlington, MA), 4 mM THPTA ligand (Lumiprobe, Hunt Valley, MD), 25 mM Ascorbic acid (MilliporeSigma, Burlington, MA), 5% DMSO (Thermo Fisher Scientific, Waltham, MA), 1× TrueBlack (Biotium, Fremont, CA), and 10 μM AZDye633 picolyl azide (Click Chemistry Tools, Scottsdale, AZ). After a 30-minute incubation at room temperature, 1 mL of 1×PBS was added to each reaction, and cells were harvested by centrifugation (10 min 10,000 g). Supernatant containing excess dye and reaction components was discarded, and the cells were washed with 1 mL of 1×PBS and pelleted again by centrifugation. Finally, each cell pellet was resuspended in 0.15 mL of 1×PBS and mixed with an equal volume of ferrofluid. All samples were run on PIPER, and cell counts were generated by image analysis using an automated bacterial image recognition algorithm.

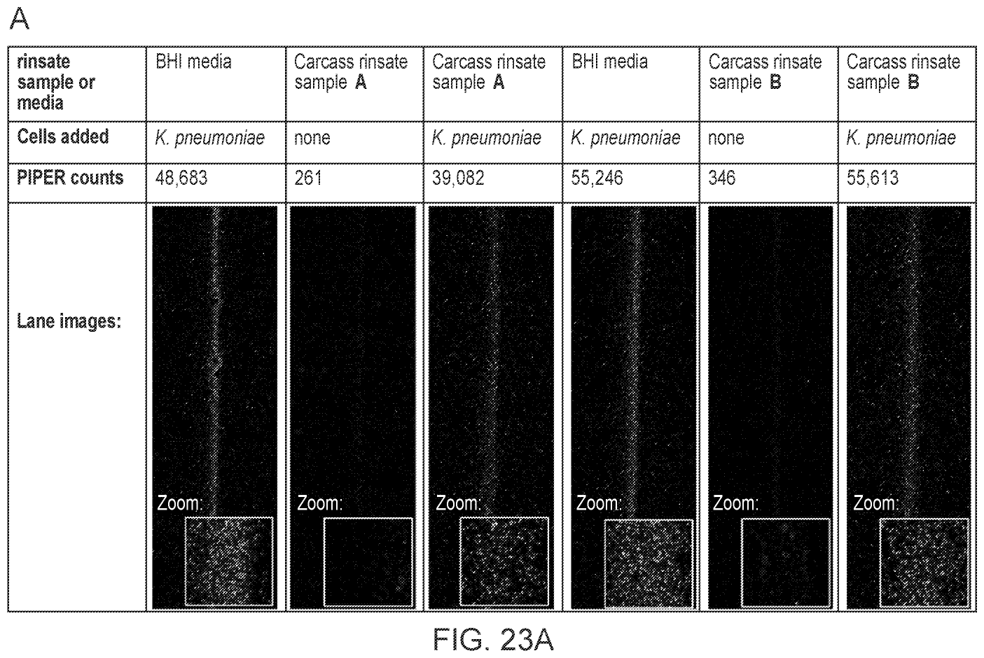

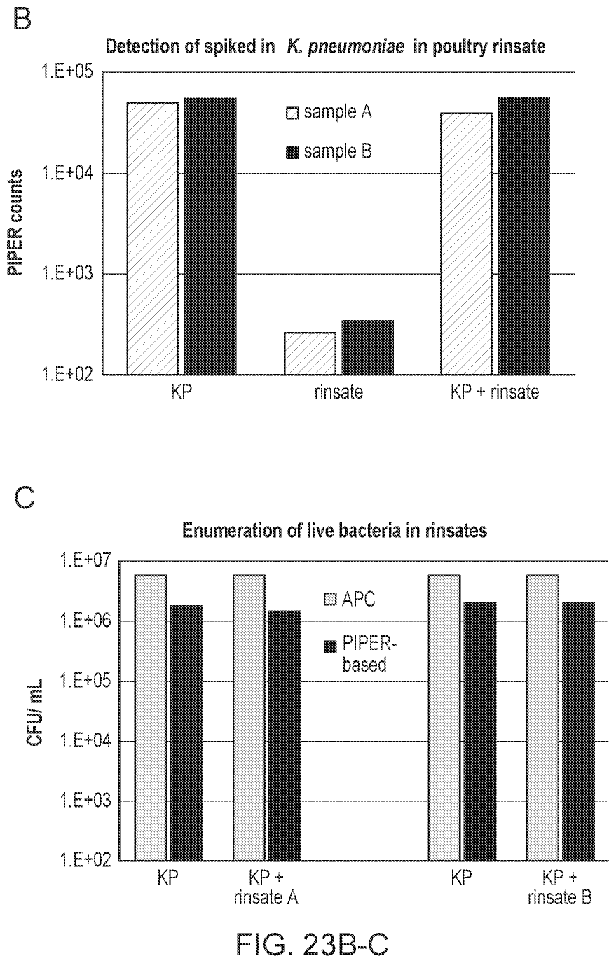

FIG. 23 depicts how 3-step metabolic labeling detects live bacteria spiked into poultry carcasses. FIG. 23A depicts PIPER images of two carcass rinsate samples or media-only controls with or without added Klebsiella pneumoniae (KP) cells subjected to 3-step metabolic labeling. FIG. 23B depicts a graph showing PIPER counts obtained from the images in FIG. 23A. Cell detection in the whole carcass rinsate samples (“KP+rinsate”) is as efficient as when cells are tested in media only (“KP”). PIPER counts in the non-spiked rinsate control samples (“rinsate”) are within system noise due to imperfections of the image algorithm that is still in development. APC of the rinsate confirmed that the innate bacterial population of the tested rinsate samples was below 100 CFU/mL. FIG. 23C depicts the enumeration of Klebsiella pneumoniae (KP) spiked into poultry rinsate samples (“KP+rinsate A”, (“KP+rinsate B”) or into media only control (“KP”). The number of live bacteria in each sample was estimated using aerobic plate count (APC, grey bars) or based on PIPER counts (black bars). To convert the PIPER counts into the number of live bacteria per swab, the values were corrected for the volume of the viewing window relative to the total sample volume loaded into the cartridge lane, and the fraction of the total eluent volume analyzed. Lower cell counts obtained by the 3-step labeling compared to APC counts could be due to imperfections of the automated image algorithm in development.

### Example 6: Detection of Innate Live Bacteria Populations in Poultry Facility Swabs Using the 3-Step Metabolic Labeling

Experimental Detail: Swab samples were collected using sponge sticks (3M, Saint Paul, MN) soaked in Letheen broth (samples G, Q) or Neutralizing buffer (sample H). Samples were stored on ice and processed within 24 hours after collection. Eluents from the sponges were aseptically extracted and the rest was frozen at −80° C. until the time of analysis. Prior to 3-step metabolic labeling, an aliquot from each eluent was removed for serial dilution and plating on APC petrifilm (3M, Saint Paul, MN) and incubated as per manufacturer instructions to evaluate the number of total viable bacteria by the “gold standard” method. Each eluent sample was divided into two aliquots for incubation with EDA or without EDA, as a control for non-specific staining with the fluorophore. The 1^{st} step of labeling was carried out by adding 5 μg/mL chloramphenicol and 1 mM EDA, where indicated, followed by 90 min incubation at 37° C. Cells were harvested by centrifugation (10 min 10,000 g), and supernatant containing media and excess EDA was discarded. The 2^{nd} step was carried out by resuspending the cells in 0.75 mL of 1×PBS and mixing them with 0.25 mL of 96% ethanol. After 5 min incubation at room temperature, cells were pelleted by centrifugation (10 min 10,000 g) and supernatant containing media with ethanol and residual EDA was discarded. The 3^{rd} step was carried out by resuspending cell pellets in 0.25 mL of 1×PBS solution containing 200 μM copper sulfate (II) (MilliporeSigma, Burlington, MA), 400 μM THPTA ligand (Lumiprobe, Hunt Valley, MD), 2.5 mM Ascorbic acid (MilliporeSigma, Burlington, MA), 5% DMSO (Thermo Fisher Scientific, Waltham, MA), and 10 μM CF488 picolyl azide fluorescent dye (Biotium, Fremont, CA) or AZDye633 picolyl azide (Click Chemistry Tools, Scottsdale, AZ). After 30-minute incubation at room temperature, 1 mL of 1×PBS was added to each reaction, and cells were harvested by centrifugation (10 min 10,000 g). Supernatant containing excess dye and reaction components was discarded, and the cells were washed with 1 mL of 1×PBS and pelleted again by centrifugation. Finally, each cell pellet was resuspended in 0.3 mL of 1×PBS and mixed with an equal volume of ferrofluid. All samples were run on PIPER in duplicate, and cell counts were generated by image analysis using an automated bacterial image recognition algorithm.

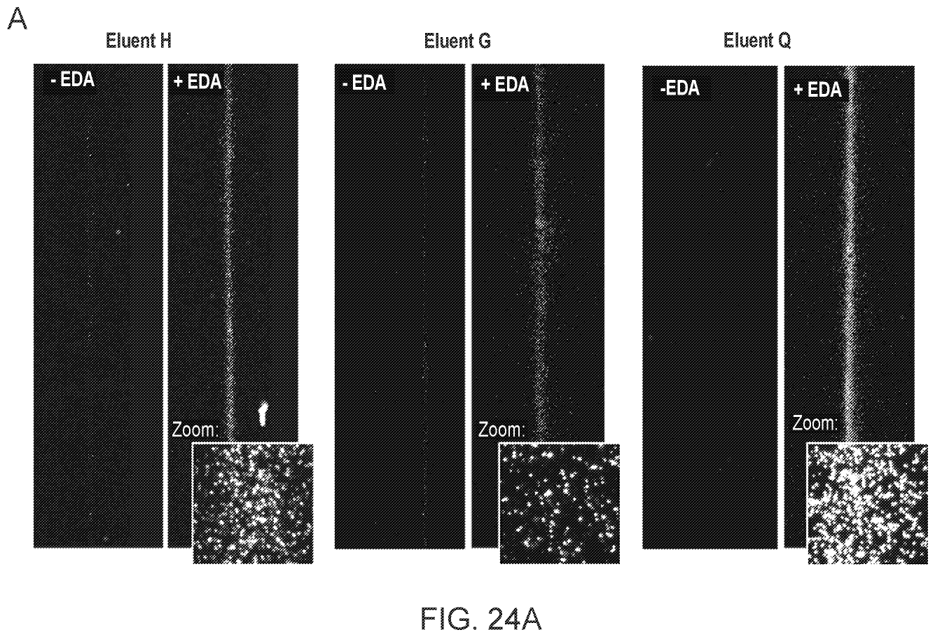

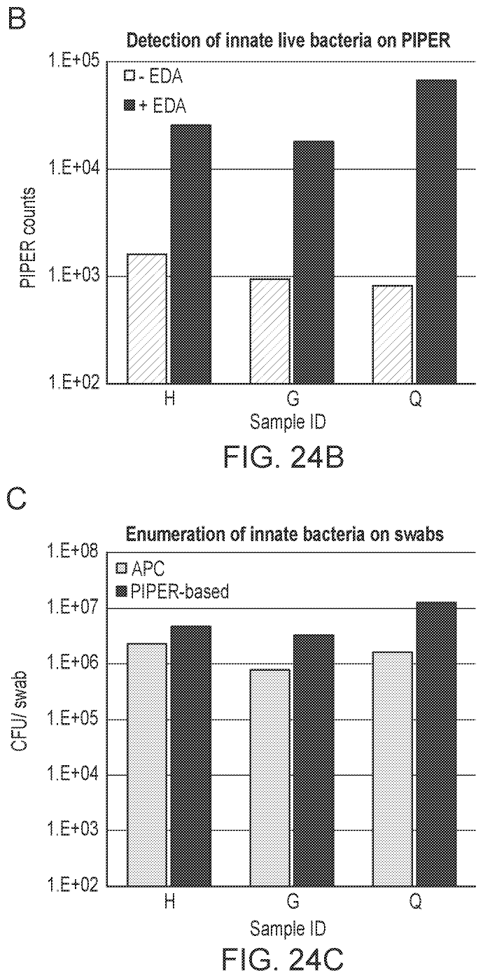

FIG. 24 depicts how 3-step metabolic labeling detects innate live bacterial populations in poultry facility swabs. FIG. 24A depicts PIPER images of innate populations of live bacteria in swabs from a poultry processing facility. Samples H and G were swabs of the manual eviscerator drain, and sample Q was a swab of the floor drains and ice maker. Omitting EDA from the 1^{st} step of labeling led to a dramatic reduction of the signal, indicating that click labeling is specific. FIG. 24B depicts a graph showing PIPER counts obtained from the images in FIG. 24A. Addition of EDA led to a ˜1.5 log increase in PIPER cell counts (black bars) over (-EDA) control (striped bars). PIPER counts in the (-EDA) control sample are within system noise due to imperfections of the image algorithm that is still in development. FIG. 24C depicts an enumeration of the live bacteria in swabs by aerobic plate count (APC) or 3-step metabolic labeling. The number of live bacteria in each sample was estimated using APCs (grey bars) or based on PIPER counts (black bars). To convert the PIPER counts into the number of live bacteria per swab, the values were corrected for the volume of the viewing window relative to the total sample volume loaded into the cartridge lane, and the fraction of the total eluent volume analyzed. Higher cell counts obtained by the 3-step labeling compared to APC counts could be due to detection of aerotolerant anaerobes or other bacteria that do not develop macroscopic colonies on APC petrifilm.

### Example 7: Detection of Innate Live Bacteria Populations in Whole Carcass Rinsates Using the 3-Step Metabolic Labeling

Experimental Detail: Whole carcass rinsate samples were prepared as per FSIS USDA guidance using two separate whole chickens from the grocery store obtained prior to “use by” date. Sodium polyanethol sulfonate was added to 0.05% to limit blood clotting. Prior to 3-step metabolic labeling, an aliquot from each rinsate was removed for serial dilution and plating on APC petrifilm (3M, Saint Paul, MN) and incubated as per manufacturer instructions to evaluate the number of total viable bacteria by the “gold standard” method. 1 mL rinsate aliquots were used for incubation with, or without EDA, as a control for non-specific staining with the fluorophore. The 1^{st} step of labeling was carried out by adding 5 μg/mL chloramphenicol and 1 mM EDA, where indicated, followed by 90 min incubation at 37° C. 5 mg/ml of α-chymotrypsin from bovine pancreas (MilliporeSigma, Burlington, MA) was added during the last 15 min of incubation to break up protein aggregates. Cells were harvested by centrifugation (10 min 10,000 g), and supernatant was discarded. The 2^{nd} step was carried out by resuspending the pellets in 0.75 mL of 1×PBS and mixing them with 0.25 mL of 96% ethanol. After a 5-minute incubation at room temperature, cells were pelleted by centrifugation (10 min 10,000 g) and supernatant containing media with ethanol and residual EDA was discarded. The 3^{rd} step was carried out by resuspending cell pellets in 0.25 mL of 1×PBS solution containing 2 mM copper sulfate (II) (MilliporeSigma, Burlington, MA), 4 mM THPTA ligand (Lumiprobe, Hunt Valley, MD), 25 mM Ascorbic acid (MilliporeSigma, Burlington, MA), 5% DMSO (Thermo Fisher Scientific, Waltham, MA), and 10 μM AZDye633 picolyl azide (Click Chemistry Tools, Scottsdale, AZ). After a 30-minute incubation at room temperature, 1 mL of 1×PBS was added to each reaction, and cells were harvested by centrifugation (10 min 10,000 g). Supernatant containing excess dye and reaction components was discarded, and the cells were washed with 1 mL of 1×PBS and pelleted again by centrifugation. Finally, each cell pellet was resuspended in 0.3 mL of 1×PBS and mixed with an equal volume of ferrofluid. All samples were run on PIPER in duplicate, and cell counts were generated by image analysis using an automated bacterial image recognition algorithm.

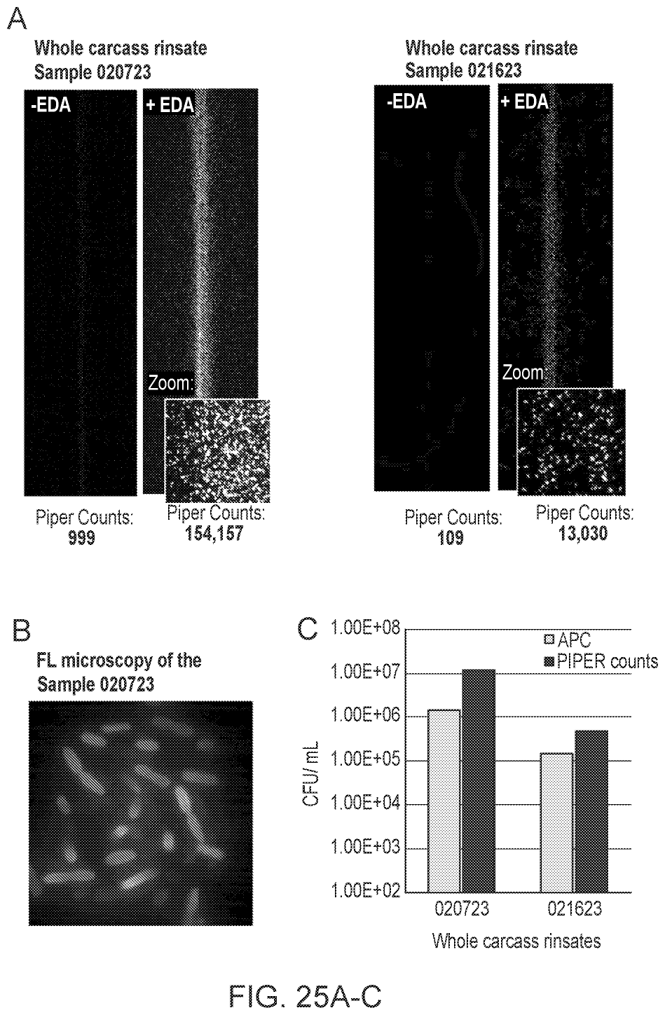

FIG. 25 depicts how 3-step metabolic labeling detects innate live bacterial populations in whole carcass rinsates. FIG. 25A depicts PIPER images of innate populations of live bacteria detected by 3-step metabolic labeling in two separate whole carcass rinsate samples. Omitting EDA from the 1^{st} step of labeling led to a dramatic reduction of the signal, indicating that click labeling is specific. FIG. 25B depicts fluorescent microscopy of whole carcass rinsate sample “020723” subjected to 3-step metabolic labeling revealed cell-like rod shapes characteristic of bacteria. FIG. 25C depicts an enumeration of the live bacteria in swabs by aerobic plate count (APC) or 3-step metabolic labeling. The number of live bacteria in each sample was estimated using APCs (grey bars) or based on PIPER counts (black bars). To convert the PIPER counts into the number of live bacteria per swab, the values were corrected for the volume of the viewing window relative to the total sample volume loaded into the cartridge lane, and the fraction of the total sample volume analyzed. Higher cell counts obtained by the 3-step labeling compared to APC counts could be due to detection of aerotolerant anaerobes or other bacteria that do not develop macroscopic colonies on APC petrifilm.

### Example 8: Detection and Enumeration of Live Bacteria in Reconstituted Swab Eluents by Aerobic Plate Count (APC) or 3-Step Metabolic Labeling

Experimental detail: Swab samples were collected using sponge sticks (3M, Saint Paul, MN) soaked in Letheen broth or Neutralizing buffer. Various areas at the pre-chill poultry processing facility, including evisceration equipment, floor drains and ice maker, were swabbed as per manufacturer instructions, and placed on ice. All samples were processed within 24 hours after collection. Eluents from the sponges were aseptically extracted, a 0.1 mL aliquot was removed for plating and individual species isolation, and the rest was frozen at −80° C. until the time of analysis. For the plating and individual species isolation, the 0.1 mL aliquot from each sample was diluted in sterile 1× PBS (phosphate buffered saline) and 0.1 mL of each dilution was spread on TSA (tryptic soy agar) plates. From each eluent, between 1 and 5 individual colonies representing different morphologies were isolated. Isolates were obtained and identified as described in the legend to Table 1. To reconstitute the eluents with simulated high levels of bacteria, up to two species isolated from each swab sample were sub-cultured in BHI at 37° C. overnight, diluted to absorbance at 600 nm (OD600) of 0.1+/−0.01, and mixed in a 1:1 ratio. If only one isolate was available from a particular eluent, it was used in double the amount as a monoculture. Cells were added to the corresponding thawed eluent in a ratio of 9:1 (v:v) eluent to cell suspensions, and incubated at 4° C. overnight to simulate cold stress and allow any components of the eluent to exert their influence on the spiked cells. On the next day, an aliquot from each eluent was removed for serial dilution and plating on APC petrifilm (3M, Saint Paul, MN) and incubated as per manufacturer instructions to evaluate the number of total viable bacteria by the “gold standard” method. The rest of the eluents were subjected to the 3-step metabolic labeling protocol. The 1^{st} step was carried out by adding 1 mM EDA and 5 μg/mL chloramphenicol to each eluent, followed by a 90-minute incubation at 37° C. Cells were harvested by centrifugation (10 min 10,000 g), and supernatant containing media and excess (unincorporated) EDA was discarded. The 2^{nd} step was carried out by resuspending the cells in 0.75 mL of 1×PBS and mixing them with 0.25 mL of 96% ethanol. After a 5-minute incubation at room temperature, cells were pelleted by centrifugation (10 min 10,000 g) and supernatant containing media with ethanol and residual EDA was discarded. The 3^{rd} step was carried out by resuspending cell pellets in 0.25 mL of 1×PBS solution containing 200 μM copper sulfate (II) (MilliporeSigma, Burlington, MA), 400 μM THPTA ligand (Lumiprobe, Hunt Valley, MD), 2.5 mM Ascorbic acid (MilliporeSigma, Burlington, MA), 10 μM CF488 picolyl azide fluorescent dye (Biotium, Fremont, CA), and 5% DMSO (Thermo Fisher Scientific, Waltham, MA). After a 30-minute incubation at room temperature, 1 mL of 1×PBS was added to each reaction, and cells were harvested by centrifugation (10 min 10,000 g). Supernatant containing excess dye and reaction components was discarded, cells were washed by 1 mL of 1×PBS, and cells were pelleted again by centrifugation. Finally, each cell pellet was resuspended in 0.3 mL of 1×PBS and mixed with an equal volume of ferrofluid. All samples were run on PIPER in duplicate, and cell counts were generated by image analysis using an automated bacterial image recognition algorithm.

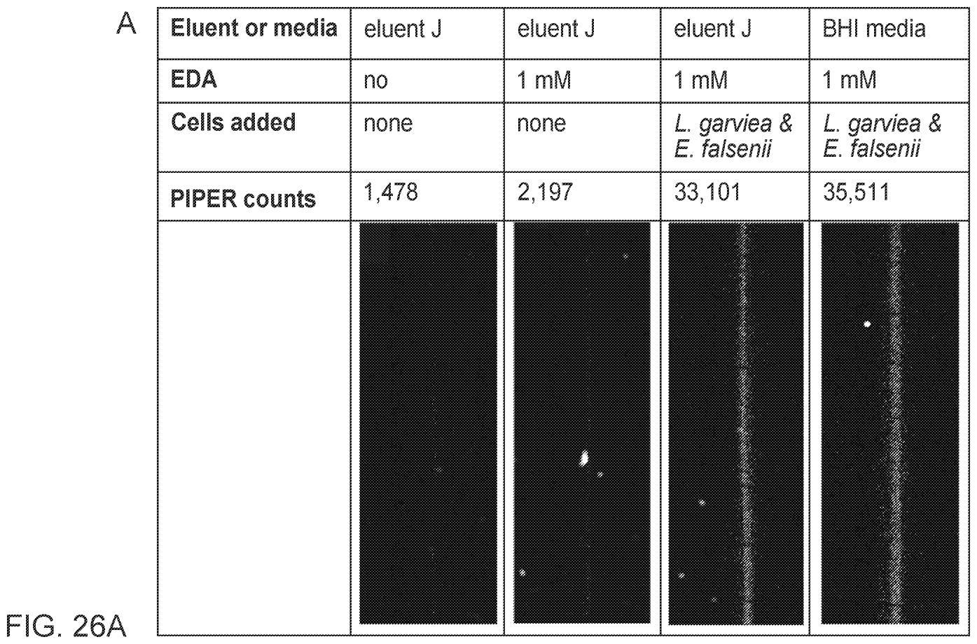

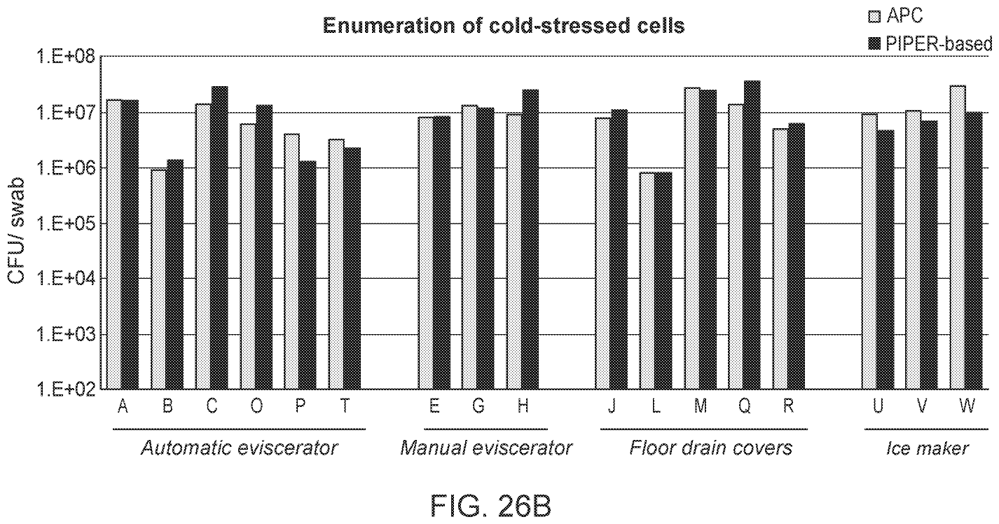

FIG. 26 depicts the detection and enumeration of live bacteria in reconstituted swab eluents by aerobic plate count (APC) or 3-step metabolic labeling. FIG. 26A depicts the detection of Lactococcus garviea and Empedobacter falsenii in a reconstituted eluent from swabbing a floor drain cover at a poultry processing facility (Sample J). The PIPER counts obtained from 3-step-metabolic labeling in the presence of eluent are very similar to those in BHI (brain heart infusion) media only, demonstrating that sample components do not interfere with cell detection (lanes 3 and 4). Eluent subjected to the 3-step labeling without added cells (lane 2) did not reveal cell detection significantly above the background (lane 1, no EDA). PIPER counts in the (no EDA) control sample are within system noise due to imperfections of the image algorithm that is still in development. The same controls were carried out for all eluents shown in FIG. 26B (data not shown). FIG. 26B depicts how the number of live bacteria in each sample was estimated using aerobic plate counts (APCs; grey bars) or based on PIPER counts (black bars). To convert the PIPER counts into the number of live bacteria per swab, the values were corrected for the volume of the viewing window relative to the total sample volume loaded into the cartridge lane, and the fraction of the total eluent volume analyzed. PIPER counts of live bacteria by the 3-step metabolic labeling method in simulated environmental swab samples fell within 0.5 log of the counts obtained by the “gold standard” aerobic plate count (APC) method. The samples are grouped according to the areas of swab collection. Each eluent was stored at −80° C. while bacterial species from those samples were isolated and identified. For this experiment, the original eluents were thawed and reconstituted as described below by adding a culture(s) of the bacteria species isolated from that exact sample. Specifically, eluent A was spiked with Brachybacterium paraconglomeratum and Staphylococcus pseudoxylosus; eluent B was spiked with Staphylococcus lloydii and an unidentified isolate B2; eluent C was spiked with Mammaliicoccus lentus; eluent O was spiked with Corynebacterium ammoniogenes and Staphylococcus pseudoxylosus; eluent P was spiked with an unidentified isolate P1; eluent T was spiked with Rothia nasimurium; eluent E was spiked with Staphylococcus nepalensis and Bacillus licheniformis; eluent G was spiked with Macrococcus cesolyticus and Acinobacter iwofii; eluent H was spiked with Pseudomonas mosselii and Exiguobacterium indicum; eluent J was spiked with Lactococcus garviea and Empedobacter falsenii; eluent L was spiked with Exiguobacterium urantiacum and Macrococcus caseolyticus; eluent M was spiked with Cytobacillus solani; eluent Q was spiked with Pseudomonas alcaliphila and an unidentified isolate Q1; eluent R was spiked with Kurthia gibsonii and Acinobacter geminorum; eluent U was spiked with Bacillus mojavensis and an unidentified isolate U1; eluent V was spiked with Microbacterium invictum and Pseudoxanthomonas mexicana; eluent W was spiked with Microbacterium maritypicum and Microbacterium testaceum.

### General Information regarding the Disclosure

While various inventive embodiments have been described and illustrated herein, those of ordinary skill in the art will readily envision a variety of other means, functionality, steps, and/or structures (including software code) for performing the functionality disclosed and/or obtaining the results and/or one or more of the advantages described herein, and each of such variations and/or modifications is deemed to be within the scope of the inventive embodiments described herein. More generally, those skilled in the art will readily appreciate that all parameters, and configurations described herein are meant to be exemplary and that the actual parameters, and configurations will depend upon the specific application or applications for which the inventive teachings is/are used. Those skilled in the art will recognize, or be able to ascertain using no more than routine experimentation, many equivalents to the specific inventive embodiments described herein. It is therefore to be understood that the foregoing embodiments are presented by way of example only and that, within the scope of any claims supported by this disclosure and equivalents thereto, inventive embodiments may be practiced otherwise than as specifically described and claimed. Inventive embodiments of the present disclosure are also directed to each individual feature, system, apparatus, device, step, code, functionality and/or method described herein. In addition, any combination of two or more such features, systems, apparatuses, devices, steps, code, functionalities, and/or methods, if such features, systems, apparatuses, devices, steps, code, functionalities, and/or methods are not mutually inconsistent, is included within the inventive scope of the present disclosure. Further embodiments may be patentable over prior art by specifically lacking one or more features/functionality/steps (i.e., claims directed to such embodiments may include one or more negative limitations to distinguish such claims from prior art).

The embodiments of the present disclosure can be implemented in any of numerous ways. For example, some embodiments may be implemented (e.g., as noted) using hardware, software or a combination thereof. When any aspect of an embodiment is implemented at least in part in software, the software code can be executed on any suitable processor or collection of processors, servers, and the like, whether provided in a single computer or distributed among multiple computers.

Some of the inventive embodiments of the present disclosure are directed to each individual feature, system, article, material, component, kit, method, and step, described herein. In addition, any combination of two or more such features, systems, articles, materials, components, kits, methods, and steps, if such features, systems, articles, materials, components, kits, methods, and steps, are not mutually inconsistent, is included within the inventive scope of the present disclosure. Some embodiments disclosed herein may also be combined with one or more features, as well as complete systems, devices or methods of other embodiments (as well as known systems, devices, or methods) to yield yet other embodiments and inventions. Moreover, some embodiments, may be distinguishable from the prior art by specifically lacking one and/or another feature disclosed in the particular prior art reference(s); i.e., claims to some embodiments may be distinguishable from the prior art by including one or more negative limitations.

Also, as shown above, various inventive concepts may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to) for a particular embodiment(s).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein, “about” means within a statistically meaningful range of a value or values such as a stated concentration, length, molecular weight, pH, sequence identity, time frame, temperature or volume. Such a value or range can be within an order of magnitude, typically within 20%, more typically within 10%, and even more typically within 5% of a given value or range. The allowable variation encompassed by “about” will depend upon the particular system under study, and can be readily appreciated by one of skill in the art.

All of the patents, patent applications, patent application publications and other publications recited herein are hereby incorporated by reference as if set forth in their entirety.

The present invention has been described in connection with what are presently considered to be the most practical and preferred embodiments. However, the invention has been presented by way of illustration and is not intended to be limited to the disclosed embodiments. Accordingly, one of skill in the art will realize that the invention is intended to encompass all modifications and alternative arrangements within the spirit and scope of the invention as set forth in the appended claims.

1. A method for quantifying total live bacteria in a sample comprising:
obtaining a sample comprising a mixture of two or more types of live bacteria;
contacting the live bacteria with at least one D-amino acid probe under conditions
sufficient for bacterial cell wall synthesis, wherein:
the bacteria covalently incorporate the at least one D-amino acid probe, the amino acid probe includes a covalently attached fluorophore;
adding the bacteria including the covalently incorporated D-amino acid probe to
an assay processing device for at least counting cells; and
detecting the labeled bacteria.

2. The method of claim 1, wherein the D-amino acid probe is a single amino acid or a dipeptide.

3. The method of claim 1, further comprising removing unincorporated D-amino acid probes from the sample.

4. The method of claim 1, wherein the labeled bacteria are detected via a fluorescence detector.

5. The method of claim 1, further comprising flowing the bacteria labeled with the D-amino acid probe toward an imaging region of the assay processing device, the imaging region including the fluorescence detector.

6. The method of claim 1, further quantifying a number of total live bacteria detected in the sample.

7. The method of claim 1, wherein the D-amino acid probe is selected from the group consisting of: HADA, BADA, NADA, FDL, TDL, HDL, NDL, FADA, TADA, HADG, NADG, FADG, and TADG.

8. A method for quantifying total live bacteria in a sample comprising: obtaining a sample comprising live bacteria;
contacting the live bacteria with at least one D-amino acid probe under conditions sufficient for bacterial cell wall synthesis, wherein:
the bacteria covalently incorporate the at least one D-amino acid probe, and the D-amino acid probe comprises a clickable bioorthogonal handle;
contacting the live bacteria with a fluorescent label comprising a bioorthogonal reactive group, wherein the clickable bioorthogonal reactive group forms a covalent bond with the clickable bioorthogonal handle; and
detecting the live bacteria.

9. The method of claim 8, wherein the D-amino acid probe comprises a single amino acid or a dipeptide.

10. The method of claim 8, wherein the at least one D-amino acid probe comprises a combination of an amino acid and a dipeptide.

11. The method of claim 8, wherein the clickable bioorthogonal reactive group comprises an azide, alkyne, or cycloalkyne group.

12. The method of claim 8, wherein the clickable bioorthogonal reactive group comprises a cycloalkyne group.

13. The method of claim 8, wherein the clickable bioorthogonal reactive group comprises an azide.

14. The method of claim 8, wherein the clickable bioorthogonal reactive group comprises an alkyne.

15. The method of claim 8, wherein the D-amino acid probe comprises an azide, alkyne, or cycloalkyne group.

16. The method of claim 8, wherein the D-amino acid probe comprises an azide group.

17. The method of claim 8, wherein the D-amino acid probe comprises an alkyne group.

18. The method of claim 8, wherein the D-amino acid probe comprises D-propargylglycine (EDA).

19. The method of claim 8, wherein the at least one D-amino acid probe comprises a combination of EDA and EDA-DA.

20. The method of claim 8, wherein the combination of EDA and EDA-DA is used at a ratio of from about 1:2, to about 2:1.

21. The method of claim 8, wherein the combination of EDA and EDA-DA is used at a ratio of about 1:1.

22. The method of claim 8, further comprising removing the unincorporated D-amino acid probe from the sample.

23. The method of claim 8, further comprising removing the unreacted fluorescent label from the sample.

24. The method of claim 8, wherein the labeled bacteria are detected via a fluorescence detector.

25. The method of claim 8, further comprising flowing the labeled bacteria including the at least one D-amino acid probe toward an imaging region of an assay device, the imaging region including the fluorescence detector.

26. The method of claim 8, further comprising quantifying the number of total live bacteria detected in the sample.

27. The method of claim 8, wherein the fluorescent label comprises an azide, alkyne, or cycloalkyne group.

28. The method of claim 8, wherein the fluorescent label includes an azide group.

29. The method of claim 8, wherein the fluorescent label comprises CF488 picolyl azide, AZDye 488 Picolyl Azide, CF633 picolyl azide, or AZDye™ 633 Azide.

30. The method of claim 8, wherein the method further comprises a fixation step.

31. The method of claim 8, wherein the fixation step comprises the addition of an organic solvent.

32. The method of claim 8, wherein the organic solvent is ethanol.

33. The method of claim 8, wherein the fixation step is followed by an incubation period.

34. The method of claim 8, wherein the incubation period is from about 1 minute, to about 30 minutes.

35. The method of claim 8, wherein the incubation period is about 1 minute, about 2 minutes, about 3 minutes, about 4 minutes, about 5 minutes, about 7 minutes, about 10 minutes, or about 15 minutes.

36. The method of claim 8, wherein the clickable bioorthogonal handle of the D-amino acid probe reacts with the clickable bioorthogonal reactive group of the fluorescent label to form a 1,2,3-triazole.

37. The method of claim 8, wherein the reaction between the D-amino acid and the fluorescent label is a copper catalyzed click chemistry reaction.

38. The method of claim 8, wherein the reaction between the D-amino acid and the fluorescent label is a copper-free reaction.

39. The method of claim 8, wherein the reaction between the D-amino acid and the fluorescent label is a copper-free strain promoted click chemistry reaction.

40. The method of claim 1, wherein the contacting the live bacteria with at least one D-amino acid probe comprises incubating the live bacteria with the at least one D-amino acid probe.

41. The method of claim 1, wherein the contacting the live bacteria with at least one D-amino acid probe occurs for between about 10 and about 120 minutes.

42. The method of claim 1, wherein the contacting the live bacteria with at least one D-amino acid probe occurs for at least about 10 minutes, at least about 20 minutes, at least about 30 minutes, at least about 40 minutes, at least about 50 minutes, at least about 60 minutes, at least about 70 minutes, at least about 80 minutes, at least about 90 minutes, at least about 100 minutes, at least about 110 minutes, or at least about 120 minutes.

43. The method of claim 1, wherein the contacting the live bacteria with at least one D-amino acid probe occurs for between about 20 and about 90 minutes.

44. The method of claim 1, wherein the contacting the live bacteria with at least one D-amino acid probe comprises incubating the live bacteria with the at least one D-amino acid probe for between about 20 and about 90 minutes.

45. The method of claim 1, wherein the sample is supplemented with a cell metabolism booster.

46. The method of claim 45, wherein the cell metabolism booster comprises at least one of glucose and sodium pyruvate.

47. The method of claim 1, wherein the sample is supplemented with a small molecule that controls cell doubling.

48. The method of claim 47, wherein the small molecule comprises DL-serine hydroxamate or chloramphenicol.

49. The method of claim 1, wherein the sample is supplemented with at least one of glucose and sodium pyruvate.

50. The method of claim 1, wherein quantifying the total live bacteria in the sample is performed using a microfluidic device.

51. The method of claim 1, wherein quantifying the total live bacteria in the sample is performed using flow cytometry.

52. The method of claim 1, wherein quantifying the total live bacteria in the sample is performed using a ferrofluid-based microfluidic device.

53. The method of claim 1, wherein the sample is obtained from a food processing plant.

54. The method of claim 1, wherein the sample is obtained from a sample from a beef or poultry processing plant.

55. The method of claim 1, wherein the sample is obtained from a poultry processing plant.

56. The method of claim 1, wherein the sample is obtained from a carcass.

57. The method of claim 1, wherein the method does not include culturing the bacteria before analysis.

58. The method of claim 1, wherein the quantifying step comprises counting individually labeled bacteria cells.

59. The method of claim 1, wherein the live bacteria sample comprises gram negative bacteria.

60. The method of claim 1, wherein the live bacteria is selected from the group consisting of: Aeromonas hydrophila, Bukholderia cenocepacia, Campylobacter jejuni, Citrobacter freundii, Enterobacter sakasakii (Cronobacter), Escherichia coli, Flavobacterium Sp., Hafnia alvei, Klebsiella pneumoniae, Kluyvera Sp., Moraxella catarrhalis, Proteus mirabilis, Pseudomonas aeruginosa, Salmonella Typhimurium, Serratia liquefaciens, Shewanella putrefaciens, Shigella flexneri, Vibrio parahaemolyticus, and Yersinia enterocolitica.

61. The method of claim 1, wherein the live bacteria sample comprises gram positive bacteria.

62. The method of claim 1, wherein the bacteria is selected from the group consisting of: Bacillus cereus, Bacteroides fragilis, Brochothrix thermosphacta, Clostridium perfringens, Corynebacterium sp, Enterococcus faecalis, Lactobacillus brevis, Lactococcus lactis, Leuconostoc lactis, Listeria monocytogenes, Staphylococcus aureus, Streptococcus pyogenes, and Weissella viridescens.

63. The method of claim 1, wherein the bacteria is selected from the group consisting of: Acinetobacter geminorum, Acinetobacter haemolyticus, Acinetobacter lwoffii, Acinetobacter pittii or A. calcoaceticus, Bacillus licheniformis, Bacillus mojavensis, Brachybacterium paraconglomeratum, Chryseobacterium gambrini, Corynebacterium ammoniagenes, Coryneobacterium callunae, Cytobacillus solani, Empedobacter falsenii, Exiguobacterium indicum, Heyndrickxia oleronia, Kocuria tytonicola; or K. tytonis, Kurthia gibsonii, Kurthia populi, Macrococcus caseolyticus, Mammaliicoccus lentus, Microbacterium invictum, Microbacterium maritypicum, Microbacterium testaceum, Pseudomonas mosselii, Pseudoxanthomonas mexicana, Rothia nasimurium, Staphylococcus pseudoxylosus, Staphylococcus lloydii; or S. kloosii, Staphylococcus nepalensis, Staphylococcus ureilyticus, and Streptococcus pluranimalium.

64. The method of claim 1, wherein the sample comprises both gram negative and gram positive bacteria.

65. The method of claim 8, wherein the method further comprises a fixation step prior to the contacting of live bacteria with a fluorescent label.

66. A quantification system configured to quantify fluorescently labeled live bacteria from a sample according to the method of claim 1, comprising:
a ferrofluidic assay device configured to receive a microfluidic cartridge containing a sample;
the microfluidic cartridge includes a plurality of microfluidic channels; each microfluidic channel contains an imaging window;
an imager configured to image each window of the cartridge either separately or together;
a controller configured to control at least one of the ferrofluidic assay device, the microfluidic cartridge containing sample mixed with ferrofluid, and the imager; and
assay processing components comprising at least one of reagents, and controls;
wherein the system is configured to at least one of moving or otherwise locating the
labeled bacteria to one or more of the windows where they can be any and all of imaged and quantified.
